SEQUENCE LISTING (96 sequences)

SEQ ID NO: 1 - DNA, 100 bp, Homo sapiens
aacaaagaaagccagggtgtcaggacaggcacagactggaacttggacccgaggcaggacagggagctggccagggaaagggtgctccaggaggagggca

SEQ ID NO: 2 - DNA, 100 bp, Homo sapiens
tgagcacagcgcccagggagagggacagacagccggctgcatgggacagcggaacccagagtgagaggggaggtggcaggacagacagacagcaggggcg

SEQ ID NO: 3 - DNA, 100 bp, Homo sapiens
ggggcagcggccccgcacccctcctccttgctgatttgcacacattggccgcttcagacacgcacttctggggccagcccctccccgcctcctccctgcc

SEQ ID NO: 4 - DNA, 100 bp, Homo sapiens
ggaggaatgtttaccagacacagagcccagagggacagcgcccagagcccggatagagagacacggcctcactggctcaggacagggggcacagccacca

SEQ ID NO: 5 - DNA, 100 bp, Homo sapiens
gagcctcctccctggggcccagagctttgtctgatcatgtgtgctggggcggggtttgtccaggaagctctgtttcctctcctctcattcctacctttgt

SEQ ID NO: 6 - DNA, 100 bp, Homo sapiens
ggcccacagcatctgaccccaggcccagctcgtcctggctggcctgggtcggcctctggagtatggtctggcgggtgccccctttcttgctccccatcct

SEQ ID NO: 7 - DNA, 100 bp, Homo sapiens
tggttttgtaatcaagactggatctaccagtgacttgctgaataaccttcggtgattcctttctcttcttgggtctcactgtatttcaaaacatgaagaa

SEQ ID NO: 8 - DNA, 100 bp, Homo sapiens
gcggtttcctgcgggtctggggtcttgcctgacttggcagtggagactgcgggcagtggagagaggaggaggtggtgtaagccctttctcatgctggtgc

SEQ ID NO: 9 - DNA, 100 bp, Homo sapiens
tgggaagagcaaaagaaaagctctatgttgcaaaacccatttttgctaacgtgtccagtgggctcccgggacgacctgtttttaaattcttggtctccct

SEQ ID NO: 10 - DNA, 100 bp, Homo sapiens
cccccggccgccccgacgggcctagtctcctctatcgctggatgaagcacgagccgggcctgggtagctatggcgacgagctgggccgggagcgcggctc

SEQ ID NO: 11 - DNA, 100 bp, Homo sapiens
ccccagtgtggtctggatgggccccagaggggcagggacagggacaggacgtggggctgtatctgacaggaacctgaggggctggcctgggaggggattg

SEQ ID NO: 12 - DNA, 100 bp, Homo sapiens
gcgtgttcgctgggggttaatgtttgccttatgaccaagtctctgtgtccgtgcctctctccattttctcttcctacctcaaacccagcaacttagaaaa

SEQ ID NO: 13 - DNA, 100 bp, Homo sapiens
ggctggcactcttcctctttttccagttcactgaggcagatgggaggcccggaggagaaagaatgaaggaaggcatttcagcccgagtaaactccccagg

SEQ ID NO: 14 - DNA, 100 bp, Homo sapiens
tggactcgcgggctccccgcaggccttccaaagtttgagcgtctcaaagcgccagcgcccctacggattagcccccagggatctctgagcctggtatcct

SEQ ID NO: 15 - DNA, 100 bp, Homo sapiens
acgcgggctctgccaccgcctgaggtcatacccaggcactgggtgttggcgggagcagtaaagcgccataaaagcaccacttggatgactattgcaaagt

SEQ ID NO: 16 - DNA, 100 bp, Homo sapiens
gataagtgacagtgagttataatcatccttgcctgtgttgtccttcccacgttaggtctgtcatgccacgtatgtccgcagtttataacaatctctatca

SEQ ID NO: 17 - DNA, 100 bp, Homo sapiens
tcgtctctaacccagctcctctatggtgcttacctcctgatccctggggcgatcagcaaaccgggtctgccaccttcttttcagagagcttaactagcag

SEQ ID NO: 18 - DNA, 100 bp, Homo sapiens
tgatattaagggcataatgaagctaagggtcagcagtcagccaagttttcgaccatcttggctttaaccagttgcggccagtttcttctgtgttacattc

SEQ ID NO: 19 - DNA, 100 bp, Homo sapiens
agctcagggaggcctcttcattcattaaaaagccacaatcaaggtttcccggcacaggattttttctgcttagagctcctccttctctaacctctctaat

SEQ ID NO: 20 - DNA, 100 bp, Homo sapiens
gaggccctcagctgtgggttctcattcgcgtgttcctaggtcacagtgacgtggacaaattgcccattccagaatacaatgggattgagaaataattggg

SEQ ID NO: 21 - DNA, 100 bp, Homo sapiens
aaaaaaaggctgccctttctagatcaggaggtccagcctctggaaacctcggagggctgcttgatctttcttttctaattcctgacaagttagaagacct

SEQ ID NO: 22 - DNA, 100 bp, Homo sapiens
actgctgcctaccctccggttccaggtatgcaggcttcctcccttctgacggttcctgctgctggagtcgtccttcctgaaaccctgcctttgcttagcc

SEQ ID NO: 23 - DNA, 100 bp, Homo sapiens
gtcaccctcctggaaactgggcctgcttggcatcaagtcagccatcagccggcccatctcctcatgctggccaaccctctgtgagtgtgtgggaggggag

SEQ ID NO: 24 - DNA, 100 bp, Homo sapiens
cccaggtgcaaccacagctctgaggccacatgggcatccccctggcaggcgtggcccacacctgcactgtctggtctgacacccagaggccctggcaaga

SEQ ID NO: 25 - DNA, 100 bp, Homo sapiens
tcttgaagagccttggtagaagtatgggcattaaaggtgattctggtgacggctcagatggaaaggagaaatatgttattgaaactggaggcaagtggta

SEQ ID NO: 26 - DNA, 100 bp, Homo sapiens
tcgctccattgtttatttgcatgtggacataagaaagggttaacatggccgacaactatttcatgagctttttggctttatttgaaaagtgaagtgtgtt

SEQ ID NO: 27 - DNA, 100 bp, Homo sapiens
aagacctgaacaccgctcccataaagccatggcttgccttggatttcagcggcacaaggctcagctgaacctggctaccaggacctggccctgcactctc

SEQ ID NO: 28 - DNA, 100 bp, Homo sapiens
ttcctagctcttttcccctgcagaagtttccaaagagactacggggctccgggagagcaggcgcttttaaatagccggcccctggctgccagccagtttg

SEQ ID NO: 29 - DNA, 100 bp, Homo sapiens
aagaaacacttctctcacccctgagctaagcagaataagaggggctggacgtgcaggactcagagtgggagcgaggagggctggggtgaggacagctttg

SEQ ID NO: 30 - DNA, 100 bp, Homo sapiens
taagagagaggcatggcagggcaaggagaggactattgaggcacacacacgtgtctggcagcctgagtgggcccagttacctggcaggcagacccatggg

SEQ ID NO: 31 - DNA, 100 bp, Homo sapiens
cccgctgctcttcctcctccttatggcttcttggttcctctatttctcgcgtcccggctccactagttggctcctgaaatactgccagggcgcacgactt

SEQ ID NO: 32 - DNA, 100 bp, Homo sapiens
ccagcacctcttccctcatctcccggccctcgagcccagatcctgacgtcgtctgatccgccagtccaggctgccccgaaggcgtgcgcggactgccggc

SEQ ID NO: 33 - DNA, 100 bp, Homo sapiens
gaacagcttctcttaaagaaagttagctgggtaggtatacagtcattgccgaggaaggcttgcacagggtgaaagctttgcttctctgctgctgtaacag

SEQ ID NO: 34 - DNA, 100 bp, Homo sapiens
aggaaccagcccaggggcttcctacagccagagggacgagctagcccgacgatggcccaggggacattgatccgtgtgaccccagagcagcccacccatg

SEQ ID NO: 35 - DNA, 100 bp, Homo sapiens
gccaacctctttcccacctcagggcctttgcacatactatttgcctctacgtggaatgttctttcctccttctcatccattagagtggcagcagtacttt

SEQ ID NO: 36 - DNA, 100 bp, Homo sapiens
caggaacacagagccattggccagccaggagggaggtagagacagaagacggtggcagcagctaccctgggtgttattttaacgtggtttgtcttggggc

SEQ ID NO: 37 - DNA, 100 bp, Homo sapiens
tcctcttcctcttctctcttctccaccttctcctcacttcgtgctctcacgcttttggacactctgtctgcccttctcctacctggggcctgatcatgac

SEQ ID NO: 38 - DNA, 100 bp, Homo sapiens
ggtgggctgtcctgaccaaacgtcccaaccctgcctgcctcatctgttccggggctgctgcctaaaccgactcacagagtgccagggctggacaggcctg

SEQ ID NO: 39 - DNA, 100 bp, Homo sapiens
ttttttacatatgcctcctcgttttgtgaattttgaaaggatgtggtttcggcctttgacatcagaggagaagctcagctatgttggctgaacgttgata

SEQ ID NO: 40 - DNA, 100 bp, Homo sapiens
caagaaatcccaacaaaagagaagaacttctccctcgaactttaaagtccgcttctttgtgttaaccaaagccagcctggcatactttgaagatcgtcat

SEQ ID NO: 41 - DNA, 100 bp, Homo sapiens
ctcccagcccacgattcccctgacccgactccctttcccagaactcagtcgcctgaacccccagcctgtggttctctcctaggcctcagcctttcctgcc

SEQ ID NO: 42 - DNA, 100 bp, Homo sapiens
tctgctcccactgcccctcttcttcctcctcggcctgctgcaagcctcacgtctgagctgtttcctgagtcacacaatgtcctggacaccctagtaatgg

SEQ ID NO: 43 - DNA, 100 bp, Homo sapiens
gttgaggcactcatggctgctgctggtgcacctgagagccttcccctaccggggaatatacttcaccagcaccactttcttcctttttttagctttttat

SEQ ID NO: 44 - DNA, 100 bp, Homo sapiens
atcattagatggcgggaaggggctttcggcagccagggtggaggagctccgaagctgacagagcagagtgggccgcctccagtgccacggggaatgaatg

SEQ ID NO: 45 - DNA, 100 bp, Homo sapiens
cctctgcagcgctccctttccctttccctttcctggttctcaaggctcccgagcttatgccttttctccttctatgctcccatcctcatcatcctgcagc

SEQ ID NO: 46 - DNA, 100 bp, Homo sapiens
tggtgatcggcgagcttggcgtgggcaagaccagcatcatcaagcgctacgtccaccagctcttctcccagcactaccgggccaccatcggggtggactt

SEQ ID NO: 47 - DNA, 100 bp, Homo sapiens
ggcacataaggcagcacattagagaaagccggcccctggatccgtctttcgcgtttattttaagcccagtcttccctgggccacctttagcagatcctcg

SEQ ID NO: 48 - DNA, 100 bp, Homo sapiens
aagaaagtaaaggaagagtggttctgcttcttagcgctagcctcaatgacgacctaagctgcacttttccccctagttgtgtcttgccatgctaaaggac

SEQ ID NO: 49 - DNA, 100 bp, Homo sapiens
ctccccagcccagtctctggtcaaactggattcctggctgttcccagaacgagctgcctttccccaccttgccacctctgcccttgttctctctgcctga

SEQ ID NO: 50 - DNA, 100 bp, Homo sapiens
gggctggcggatccctctggaggagatgcctgggtgaaagaatcctgttcggagtcagtgcctaaaagagccagtcggctctgagctgctttttattgcg

SEQ ID NO: 51 - DNA, 100 bp, Homo sapiens
accccgccgcccccgcgctgcaaactgtgtaagagggagaggaattccccgccaagttgaaaagttgaacctgcctcccaaactttctcctgtagtccag

SEQ ID NO: 52 - DNA, 100 bp, Homo sapiens
tcctgctgcagatggcaaccatcttgggcatggtgcccgcttggcatagcgcccggctccggatcttcctgtgcctggggcctcgggaggcgcctggggc

SEQ ID NO: 53 - DNA, 100 bp, Homo sapiens
cacaggatgcgagaagcctgctcgcggccttggctcattggctgggccgcggtcacctgggccgtgatgtcacggccttttagaagatcttgtggctgcc

SEQ ID NO: 54 - DNA, 100 bp, Homo sapiens
ggctggggaacccgaggcggaggaggaaggggactttgtgaacagtgggcggggagacgcagaggcagaggccctggcacgcagcgccaacgccctggtt

SEQ ID NO: 55 - DNA, 100 bp, Homo sapiens
agactctttataaatacagcttgactcagccactgtatgactgactccccggggacatgaggtggatactgttcattggggcccttattgggtccagcat

SEQ ID NO: 56 - DNA, 100 bp, Homo sapiens
ccattcttagggctataggtttaatttatttggttgtggacgtcagagccgtcatggtaagaaggaagcaaagccttttgtaataattaaagccttcaga

SEQ ID NO: 57 - DNA, 100 bp, Homo sapiens
gttgtccctgccagaggtgcagcactccgggaatgtccctcactctccccgtccctctgtcttgcccaatcctgaccaggtgcagaaatcttgccaagtg

SEQ ID NO: 58 - DNA, 100 bp, Homo sapiens
tctgtcttgcccaatcctgaccaggtgcagaaatcttgccaagtgtttccgcaggagttgctggcaattgcctcacattcctggccttggcaaagaatga

SEQ ID NO: 59 - DNA, 100 bp, Homo sapiens
tgcccagcgctcccagggtcacccctctctctagactcactttctgccccgtcaccccactgtacacccttggtcccagccccttccagtggctcagctt

SEQ ID NO: 60 - DNA, 100 bp, Homo sapiens
agatgagccaaagcccttccttcctccagtcagcctggatcctctcatccggcagaactgtcgccttgcttctctgaagcggtgaatgccctggggctgg

SEQ ID NO: 61 - DNA, 100 bp, Homo sapiens
gagaaatagaaaagtgatggcttttatttgtgaggctggcctcagcacgcggcccaagaaacagaactgaaagcggttgcagtgggcgtggccaggaggg

SEQ ID NO: 62 - DNA, 100 bp, Homo sapiens
ttggtggcctggttgtctatctgatagggcggagccttcacccttgcagcgagctctctcacaccagatgtgctctgcgtggaatcctaggccatcaggg

SEQ ID NO: 63 - DNA, 100 bp, Homo sapiens
cagctacctcccccgcatgcatgtctgtctgctgggcaaggcccaattccgaggtgacagctcaccgggcctcacccacaagtctcttccaagcattagc

SEQ ID NO: 64 - DNA, 100 bp, Homo sapiens
gattcaatcaatggtagtcagtattttcaaaaagttcctgggcccaggccgcctcctgatagaggccccgacttaggacacaaaccgctcccacgccgtt

SEQ ID NO: 65 - DNA, 100 bp, Homo sapiens
gagtcccggtactcacaggggggacgaggggaaacccttccattttgcacgcctgtaacatccagccgggctccgagtcggtcagatcccctgcctcggt

SEQ ID NO: 66 - DNA, 100 bp, Homo sapiens
ttatcgaagggcctgccgctgggagatagtccccttggggtgacatcaccgccccaacccgtttgcataaatctcttgcgctacatacaggaagtctctg

SEQ ID NO: 67 - DNA, 100 bp, Homo sapiens
ccgatccggtaatgggcctgggagatgccagattagcgtggtgcctgtccggagagacgggccagctgatgcccaggtcggggccctgccgctggccaca

SEQ ID NO: 68 - DNA, 100 bp, Homo sapiens
caggaaaggccttggaaatctgcacatggagtaagagcagaaatggaagcgtcttcagggagaacatgatcttctcttcaaactctacccctcctggctt

SEQ ID NO: 69 - DNA, 100 bp, Homo sapiens
tttgctaattacttccaaaagcctcccatctgtcatcccacccagactgcgcgcttctaattcctcctaccccacatgctgtgcccaatgaaaagtatgg

SEQ ID NO: 70 - DNA, 100 bp, Homo sapiens
tgcccaatgaaaagtatggtcagcgagcgaaggtttgcaaggagacagacgagggcgaaattaagccaggcggcttccctttaaatcctcgcaaagcaga

SEQ ID NO: 71 - DNA, 100 bp, Homo sapiens
gcagccaggttaggccaggaggaccatgtgaatggggccagagggctcccgggctgggcaggtaaggagcgctggtattgggggcgcaggcgccggggtg

SEQ ID NO: 72 - DNA, 100 bp, Homo sapiens
gtccccccaccctgccccactgttgatcctggctctgccaccaatcatgcgacatcaggcaactcctctcctaagcctctgttggttccttgtttattaa

SEQ ID NO: 73 - DNA, 100 bp, Homo sapiens
aggaactgggctgttagggattttccttaggccctttggtttccgcctacggagaggtttcccccattggttgctcttcctcagccagggttacttcctg

SEQ ID NO: 74 - DNA, 100 bp, Homo sapiens
accactgcaactgggtcttgcagtggggaagagggactgggctcaactccgaatacagcgtgggcaagagggaatttatagccaaccagcagtatggagt

SEQ ID NO: 75 - DNA, 100 bp, Homo sapiens
ggatagcatgcaaacgcccttgagtgaaaaagcccacagagcagtgagacgagtaaatagaagctctaggacattttgtaaagcacaggggtggaggtga

SEQ ID NO: 76 - DNA, 100 bp, Homo sapiens
aatgactgcctacaagagatgacagcctatcagagatgctacagcaagtcgatattcagtcattttcaaccacaaacaagtactattaaaaagtcatact

SEQ ID NO: 77 - DNA, 100 bp, Homo sapiens
agccttttaaaatttttcttgcaaatgaccagaaagcaaggaaagaatgcggttaaaagaacaatttggtgaggaagtccttcatcagagttggttagta

SEQ ID NO: 78 - DNA, 100 bp, Homo sapiens
cggggacggaggagaggctgtgggagaagggagggaccagaggagagagcgagagagggaaccagaccccagttcgccgactaagcagaagaaagatcaa

SEQ ID NO: 79 - DNA, 100 bp, Homo sapiens
ccaggcacacagttcagggctggaaaagttcaacaagtgcatggaacatcggaaacctcctgaaaatgctaaatttgccccgagatgtcccgaagtccgg

SEQ ID NO: 80 - DNA, 100 bp, Homo sapiens
gcctggcaccgggaccatcctccgcctcaactttgcagcgtacttggaccgctctggccgccctgggcgctacccgcagagataagggcccctccctgcg

SEQ ID NO: 81 - DNA, 100 bp, Homo sapiens
cctttggaaataaacacgaaggttcacttgaagacttgggggagaatcacggtcaacttgtgacgcttggtttttcagatattcagctgctctggagagc

SEQ ID NO: 82 - DNA, 100 bp, Homo sapiens
agaagttcctgaaaccagctgcagtccagcttctctccccgagctctgtcgttaatggctcagcctctgacaggcccgggggctggggattgcaacacct

SEQ ID NO: 83 - DNA, 100 bp, Homo sapiens
tggtgatgtagctgctgggaggacagagctccgagtcacgtggcttgggcgggcctccccttcctggtgtccacagaagcccaacgtcactagctggggt

SEQ ID NO: 84 - DNA, 100 bp, Homo sapiens
ggccgctaccgcatccgcgtggccaccggggcctggctcttctccgggtcgtacaaccgcgtgcagctttggctggtcgggacgcgcggggaggcggagc

SEQ ID NO: 85 - DNA, 100 bp, Homo sapiens
gggaggtggggctggagccaggggaccacctgtgtctcattagtcctgtcgggcaaagtactgcagacgttaactccctgctggctccaactgttccctg

SEQ ID NO: 86 - DNA, 100 bp, Homo sapiens
cgggactcttgatcttcgctcgtggtactgtctgttcggctgtcttccccgcctctccccaggcacctgcatcctcccttggcacctgctgccaggctag

SEQ ID NO: 87 - DNA, 100 bp, Homo sapiens
atctggacacacagggctcccccccgcctctgacttctctgtccgaagtcgggacaccctcctaccacctgtagagaagcgggagtggatctgaaataaa

SEQ ID NO: 88 - DNA, 100 bp, Homo sapiens
tgtccccgagagggcctcaggtggtagggaacagcatgtctcctgagcccgctctgtccccagccctgcagctgctgctgtggcacagtgcactctggac

SEQ ID NO: 89 - DNA, 100 bp, Homo sapiens
gtcttctccccttctagggagaggccatgccctctcccctcaagtctgtcgctgacttcctctggcccttcccctcatgacgttttccctgctctgctgc

SEQ ID NO: 90 - DNA, 100 bp, Homo sapiens
acccaaatgtggcaatgggtttgtatccagccaccgacaggctgcatgacggtggcaaagtcacttcccctctctggcctttgtttttccacttgtaaaa

SEQ ID NO: 91 - DNA, 100 bp, Homo sapiens
ggttccagggaattgtgtaacccaatactcactgctcccctcttcattacgtattctgtgcattgcccatagaccaggcagatggagaaacaggaattct

SEQ ID NO: 92 - DNA, 100 bp, Homo sapiens
aaattgctctggtcacttctcccggcgcctgcagagagaccggctgtggcgctggtcaggtaatggcagccatggctggaaaccgggaacaatggggcct

SEQ ID NO: 93 - DNA, 100 bp, Homo sapiens
gtttagtttctttagggaaaaaacaaggcacaagtgacatttgccttggcgttcttgaccctccctctgtctcgcctgggtttgggggcccttctcatgg

SEQ ID NO: 94 - DNA, 100 bp, Homo sapiens
tggggtacagggtgaagaagggctggggccagcccaggacagaggaaggcgaggcaggcacgcaggaactgggctttttaaacccttaagcccaaggaaa

SEQ ID NO: 95 - DNA, 100 bp, Homo sapiens
gggtgggaggcttccgagagccgcctctcccggggcataaggaaggaagcggggctgcaggtaccgcctggggttcacagcaggggacgaggtgcctccc

SEQ ID NO: 96 - DNA, 100 bp, Homo sapiens
agctgactcatgaaattgctatcagaaaagcaaactgcttcccctctttcgccatcagactcatggttctgcttttcgtttatttgctgtaccttttctg

(19) United States
(12) Patent Application Publication
(10) Pub. No.: US 2013/0244902 A1
(43) Pub. Date: Sep. 19, 2013

(54) EARLY DETECTION OF PANCREATIC CANCER

(71) Applicants: Stephen N. Thibodeau, Rochester, MN (US); Liang Wang, Hartland, WI (US); Ann L. Oberg, Pine Island, MN (US); Gloria M. Petersen, Rochester, MN (US); William R. Bamlet, Hayfield, MN (US); Mariza de Andrade, Rochester, MN (US)

(72) Inventors: Stephen N. Thibodeau, Rochester, MN (US); Liang Wang, Hartland, WI (US); Ann L. Oberg, Pine Island, MN (US); Gloria M. Petersen, Rochester, MN (US); William R. Bamlet, Hayfield, MN (US); Mariza de Andrade, Rochester, MN (US)

(73) Assignee: MAYO FOUNDATION FOR MEDICAL EDUCATION AND RESEARCH, Rochester, MN (US)

(21) Appl. No.: 13/989,718

(22) PCT Filed: Nov. 22, 2011
(86) PCT No.: PCT/US11/61897

§ 371 Date: May 24, 2013

Related U.S. Application Data
(60) Provisional application No. 61/417,066, filed on Nov. 24, 2010.

(51) Int. Cl. C12Q 1/68

(52) U.S. Cl. CPC C12Q 1/6886
USPC 506/9; 435/6.11

(57) ABSTRACT

This document provides methods and materials involved in the early detection of pancreatic cancer. For example, this document provides methods and materials for assessing nucleic acid obtained from a blood sample of a human for a CpG methylation site profile that, at least in part, indicates that the human has pancreatic cancer.

### CROSS-REFERENCE TO RELATED APPLICATIONS

This application claims the benefit of U.S. Provisional Application Serial No. 61/417,066, filed Nov. 24, 2010. The disclosure of the prior application is considered part of (and are incorporated by reference in) the disclosure of this application.

### STATEMENT AS TO FEDERALLY SPONSORED RESEARCH

This invention was made with government support under grant CA102701 awarded by the National Institutes of Health. The government has certain rights in the invention.

### BACKGROUND

1. Technical Field

This document relates to methods and materials involved in the early detection of pancreatic cancer. For example, this document provides methods and materials for assessing nucleic acid obtained from a blood sample of a human for a CpG methylation site profile that, at least in part, indicates that the human has pancreatic cancer.

2. Background Information

Pancreatic cancer (PaC) is the 10th most common tumor type for men and women in yearly incidence in the United States and the fourth leading cause of cancer mortality (Jemal et al., CA Cancer J. Clin., 60(5):277-300 (2010)). PaC is associated with a very poor prognosis as it remains one of the most difficult tumors to treat. Much of this may be attributed to the late stage at which cancer is usually detected. Between 1999 and 2006, only 8% of patients were diagnosed, often by incidental finding on radiologic imaging, at a localized stage where immediate surgical resection and subsequent cure could be considered.

### SUMMARY

This document relates to methods and materials involved in the early detection of pancreatic cancer. For example, this document provides methods and materials for assessing nucleic acid obtained from a blood sample of a human for a CpG methylation site profile that, at least in part, indicates that the human has pancreatic cancer.

As described herein, nucleic acid from blood cells of humans with pancreatic cancer can contain different levels of the methylation CpG sites listed in Table 1 or 5 when compared to the level of methylation of those CpG sites in nucleic acid from blood cells of humans without pancreatic cancer. In particular, the methylation change in at least three methylation CpG sites listed in Table 1 or 5 (e.g., IL10_P348, LCN2_P86, ZAP70_P220, AIM2_P624, and TAL1_P817 CpG methylation sites) can indicate that a human has pancreatic cancer. In some cases, detecting a reduction or low level of methylation of the LCN2_P86 site can indicate that the human has resectable pancreatic cancer.

The methods and materials provided herein can allow clinicians to detect humans with pancreatic cancer at an early stage without the need to obtain invasive tissue biopsies (e.g., pancreas tissue biopsies). Such an early detection can allow patients to be treated sooner with the hopes that a successful treatment outcome will be achieved.

In general, one aspect of this document features a method for identifying a human as having pancreatic cancer. The method comprises, or consists essentially of, (a) determining whether or not nucleic acid obtained from a blood sample of a human comprises at least three methylation CpG sites that have an altered methylation status indicative of pancreatic cancer, wherein the at least three methylation CpG sites are selected from the group consisting of IL10_P348, LCN2_P86, ZAP70_P220, AIM2_P624, and TAL1_P817 CpG methylation sites, and (b) classifying the human as having pancreatic cancer if the nucleic acid comprises the at least three methylation CpG sites that have an altered methylation status indicative of pancreatic cancer, and classifying the human as not having pancreatic cancer if the nucleic acid does not comprise the at least three methylation CpG sites that have an altered methylation status indicative of pancreatic cancer. The blood sample can be a blood sample obtained from a human not subjected to a prior pancreas tissue biopsy. The method can comprise determining whether or not nucleic acid obtained from the blood sample comprises at least four methylation CpG sites that have an altered methylation status indicative of pancreatic cancer. The at least four methylation CpG sites can be selected from the group consisting of IL10_P348, LCN2_P86, ZAP70_P220, AIM2_P624, and TAL1_P817 CpG methylation sites. The method can comprise determining whether or not nucleic acid obtained from the blood sample comprises at least five methylation CpG sites that have an altered methylation status indicative of pancreatic cancer. The at least five methylation CpG sites can be selected from the group consisting of IL10_P348, LCN2_P86, ZAP70_P220, AIM2_P624, and TAL1_P817 CpG methylation sites.

In another aspect, this document features a method for identifying a human as having pancreatic cancer. The method comprises, or consists essentially of, (a) detecting the presence of at least three methylation CpG sites that have an altered methylation status indicative of pancreatic cancer in nucleic acid obtained from a blood sample of a human, wherein the at least three methylation CpG sites are selected from the group consisting of IL10_P348, LCN2_P86, ZAP70_P220, AIM2_P624, and TAL1_P817 CpG methylation sites, and (b) classifying the human as having pancreatic cancer based at least in part on the presence of the at least three methylation CpG sites that have an altered methylation status indicative of pancreatic cancer. The blood sample can be a blood sample obtained from a human not subjected to a prior pancreas tissue biopsy. The method can comprise detecting the presence of at least four methylation CpG sites that have an altered methylation status indicative of pancreatic cancer in the nucleic acid. The at least four methylation CpG sites can be selected from the group consisting of IL10_P348, LCN2_P86, ZAP70_P220, AIM2_P624, and TAL1_P817 CpG methylation sites. The method can comprise detecting the presence of at least five methylation CpG sites that have an altered methylation status indicative of pancreatic cancer in the nucleic acid. The at least five methylation CpG sites can be selected from the group consisting of IL10_P348, LCN2_P86, ZAP70_P220, AIM2_P624, and TAL1_P817 CpG methylation sites.

In another aspect, this document features a method for identifying a human as having resectable pancreatic cancer. The method comprises, or consists essentially of, (a) determining whether or not nucleic acid obtained from a blood sample of a human comprises hypomethylation of an LCN2_P86 methylation CpG site, and (b) classifying the human as having resectable pancreatic cancer if the nucleic acid comprises the hypomethylation of the LCN2_P86 methylation CpG site, and classifying the human as not having resectable pancreatic cancer if the nucleic acid does not comprise the hypomethylation of the LCN2_P86 methylation CpG site.

In another aspect, this document features a method for identifying a human as having resectable pancreatic cancer. The method comprises, or consists essentially of, (a) detecting hypomethylation of an LCN2_P86 methylation CpG site of nucleic acid obtained from a blood sample of a human, and (b) classifying the human as having resectable pancreatic cancer based at least in part on the hypomethylation.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1: Methylation level agreement between phase I and phase II. Representative Bland-Altman graph in one subject demonstrates good agreement between phase I and phase II data in most 96 CpG sites. Each dot represents one CpG site. Mean methylation level for each CpG site (from 0 to 100%) is shown in x-axis. Methylation level difference for each CpG site between phase I and phase II is shown in y-axis. The dashed lines indicate 95% confidence interval for the difference between the two assays, and the solid line indicates the average differences between the two assays.

FIG. 2: Validation of 96 selected CpG sites. Scatter plot shows reproducible methylation differences between phase I and phase II. Wilcoxon Rank Sum z-values were plotted on x-axis (phase I) and y-axis (phase II). 88 of the 96 CpG sites were validated by p value (<0.05) and direction (hyper/hypo-methylation). Although 8 CpG sites were not statistically significant, the trends in both phases are all the same.

### DETAILED DESCRIPTION

This document provides methods and materials involved in the early detection of pancreatic cancer. For example, this document provides methods and materials for assessing nucleic acid obtained from a blood sample of a human for a CpG methylation site profile that, at least in part, indicates that the human has pancreatic cancer.

As described herein, nucleic acid from blood samples of humans with pancreatic cancer can contain different levels of methylation at particular CpG sites (e.g., the methylation CpG sites listed in Table 1 or the methylation CpG sites listed in Table 5) when compared to nucleic acid from blood samples of humans without pancreatic cancer. The methylation level change in these methylated CpG sites can be used to identify humans with pancreatic cancer. For example, the methylation level changes in at least three (e.g., at least four, at least five, at least six, at least seven, at least eight, at least nine, or at least ten) methylation CpG sites listed in Table 1 or Table 5 can indicate that a human has pancreatic cancer. Methylation level changes in these methylation CpG sites listed in Table 1 can indicate that a human has pancreatic cancer. In some cases, a reduction in the level of methylation at the LCN2_P86 site for a human with pancreatic cancer, as compared to the level observed in healthy humans, can indicate that the human has resectable pancreatic cancer.

TABLE 1
Selected CpG sites.

| | | | | | Methylation | |
|---|---|---|---|---|---|---|
| | Gene | GenBank ® | GenBank ® | | change in | SEQ |
| Illumina ID | Symbol | Accession No. | GI No. | Sequence of CpG region | cancer patients | ID NO: |
| JAK3_P1075_R | JAK3 | NM_000215.2 | 47157314 | AACAAAGAAAGCCAGGGTGTCA | hypomethylation | 1 |
| | | | | GGACAGGCACAGACTGGAACTT | | |
| | | | | GGACC[CG]AGGCAGGACAGGG | | |
| | | | | AGCTGGCCAGGGAAAGGGTGCT | | |
| | | | | CCAGGAGGAGGGCA | | |
| SLC5A5_E60_F | SLC5A5 | NM_000453.1 | 4507034 | TGAGCACAGCGCCCAGGGAGAG | hypomethylation | 2 |
| | | | | GGACAGACAGCCGGCTGCATGG | | |
| | | | | GACAG[CG]GAACCCAGAGTGA | | |
| | | | | GAGGGGAGGTGGCAGGACAGAC | | |
| | | | | AGACAGCAGGGGCG | | |
| HPN_P374_R | HPN | NM_182983.1 | 33695154 | GGGGCAGCGGCCCCGCACCCCT | hypomethylation | 3 |
| | | | | CCTCCTTGCTGATTTGCACACA | | |
| | | | | TTGGC[CG]CTTCAGACACGCA | | |
| | | | | CTTCTGGGGCCAGCCCCTCCCC | | |
| | | | | GCCTCCTCCCTGCC | | |
| AXL_E61_F | AXL | NM_021913.2 | 21536465 | GGAGGAATGTTTACCAGACACA | hypomethylation | 4 |
| | | | | GAGCCCAGAGGGACAGCGCCCA | | |
| | | | | GAGCC[CG]GATAGAGAGACAC | | |
| | | | | GGCCTCACTGGCTCAGGACAGG | | |
| | | | | GGGCACAGCCACCA | | |
| CEACAM1_P44_R | CEACAM1 | NM_001712.3 | 68161539 | GAGCCTCCTCCCTGGGGCCCAG | hypomethylation | 5 |
| | | | | AGCTTTGTCTGATCATGTGTGC | | |
| | | | | TGGGG[CG]GGGTTTGTCCAGG | | |
| | | | | AAGCTCTGTTTCCTCTCCTCTC | | |
| | | | | ATTCCTACCTTTGT | | |
| TIE1_E66_R | TIE1 | NM_005424.2 | 31543809 | GGCCCACAGCATCTGACCCCAG | hypomethylation | 6 |
| | | | | GCCCAGCTCGTCCTGGCTGGCC | | |
| | | | | TGGGT[CG]GCCTCTGGAGTAT | | |
| | | | | GGTCTGGCGGGTGCCCCCTTTC | | |
| | | | | TTGCTCCCCATCCT | | |
| PI3_P274_R | PI3 | NM_002638.2 | 31657130 | TGGTTTTGTAATCAAGACTGGA | hypomethylation | 7 |
| | | | | TCTACCAGTGACTTGCTGAATA | | |
| | | | | ACCTT[CG]GTGATTCCTTTCT | | |
| | | | | CTTCTTGGGTCTCACTGTATTT | | |
| | | | | CAAAACATGAAGAA | | |
| MMP9_P189_F | MMP9 | NM_004994.2 | 74272286 | GCGGTTTCCTGCGGGTCTGGGG | hypomethylation | 8 |
| | | | | TCTTGCCTGACTTGGCAGTGGA | | |
| | | | | GACTG[CG]GGCAGTGGAGAGA | | |
| | | | | GGAGGAGGTGGTGTAAGCCCTT | | |
| | | | | TCTCATGCTGGTGC | | |
| IFNGR2_P377_R | IFNGR2 | NM_005534.2 | 47419933 | TGGGAAGAGCAAAAGAAAAGCT | hypomethylation | 9 |
| | | | | CTATGTTGCAAAACCCATTTTT | | |
| | | | | GCTAA[CG]TGTCCAGTGGGCT | | |
| | | | | CCCGGGACGACCTGTTTTTAAA | | |
| | | | | TTCTTGGTCTCCCT | | |
| HIC_1_SEQ_48_S103_R | HIC1 | NM_006497.2 | 61676185 | CCCCCGGCCGCCCCGACGGGCC | hypomethylation | 10 |
| | | | | TAGTCTCCTCTATCGCTGGATG | | |
| | | | | AAGCA[CG]AGCCGGGCCTGGG | | |
| | | | | TAGCTATGGCGACGAGCTGGGC | | |
| | | | | CGGGAGCGCGGCTC | | |
| MPL_P62_F | MPL | NM_005373.1 | 4885490 | CCCCAGTGTGGTCTGGATGGGC | hypomethylation | 11 |
| | | | | CCCAGAGGGGCAGGGACAGGGA | | |
| | | | | CAGGA[CG]TGGGGCTGTATCT | | |
| | | | | GACAGGAACCTGAGGGGCTGGC | | |
| | | | | CTGGGAGGGGATTG | | |
| TAL1_P817_F | TAL1 | NM_003189.1 | 4507362 | GCGTGTTCGCTGGGGGTTAATG | hypomethylation | 12 |
| | | | | TTTGCCTTATGACCAAGTCTCT | | |
| | | | | GTGTC[CG]TGCCTCTCTCCAT | | |
| | | | | TTTCTCTTCCTACCTCAAACCC | | |
| | | | | AGCAACTTAGAAAA | | |
| DHCR24_P652_R | DHCR24 | NM_014762.2 | 56790943 | GGCTGGCACTCTTCCTCTTTTT | hypomethylation | 13 |
| | | | | CCAGTTCACTGAGGCAGATGGG | | |
| | | | | AGGCC[CG]GAGGAGAAAGAAT | | |
| | | | | GAAGGAAGGCATTTCAGCCCGA | | |
| | | | | GTAAACTCCCCAGG | | |
| EPHA2_P203_F | EPHA2 | NM_004431.2 | 32967310 | TGGACTCGCGGGCTCCCCGCAG | hypomethylation | 14 |
| | | | | GCCTTCCAAAGTTTGAGCGTCT | | |
| | | | | CAAAG[CG]CCAGCGCCCCTAC | | |
| | | | | GGATTAGCCCCCAGGGATCTCT | | |
| | | | | GAGCCTGGTATCCT | | |
| GFI1_P208_R | GFI1 | NM_005263.2 | 71037376 | ACGCGGGCTCTGCCACCGCCTG | hypomethylation | 15 |
| | | | | AGGTCATACCCAGGCACTGGGT | | |
| | | | | GTTGG[CG]GGAGCAGTAAAGC | | |
| | | | | GCCATAAAAGCACCACTTGGAT | | |
| | | | | GACTATTGCAAAGT | | |
| GSTM2_P453_R | GSTM2 | NM_000848.2 | 23065549 | GATAAGTGACAGTGAGTTATAA | hypomethylation | 16 |
| | | | | TCATCCTTGCCTGTGTTGTCCT | | |
| | | | | TCCCA[CG]TTAGGTCTGTCAT | | |
| | | | | GCCACGTATGTCCGCAGTTTAT | | |
| | | | | AACAATCTCTATCA | | |
| AIM2_E208_F | AIM2 | NM_004833.1 | 4757733 | TCGTCTCTAACCCAGCTCCTCT | hypermethylation | 17 |
| | | | | ATGGTGCTTACCTCCTGATCCC | | |
| | | | | TGGGG[CG]ATCAGCAAACCGG | | |
| | | | | GTCTGCCACCTTCTTTTCAGAG | | |
| | | | | AGCTTAACTAGCAG | | |
| AIM2_P624_F | AIM2 | NM_004833.1 | 4757733 | TGATATTAAGGGCATAATGAAG | hypomethylation | 18 |
| | | | | CTAAGGGTCAGCAGTCAGCCAA | | |
| | | | | GTTTT[CG]ACCATCTTGGCTT | | |
| | | | | TAACCAGTTGCGGCCAGTTTCT | | |
| | | | | TCTGTGTTACATTC | | |
| IL10_P85_F | IL10 | NM_000572.2 | 24430216 | AGCTCAGGGAGGCCTCTTCATT | hypomethylation | 19 |
| | | | | CATTAAAAAGCCACAATCAAGG | | |
| | | | | TTTCC[CG]GCACAGGATTTTT | | |
| | | | | TCTGCTTAGAGCTCCTCCTTCT | | |
| | | | | CTAACCTCTCTAAT | | |
| IL10_P348_F | IL10 | NM_000572.2 | 24430216 | GAGGCCCTCAGCTGTGGGTTCT | hypomethylation | 20 |
| | | | | CATTCGCGTGTTCCTAGGTCAC | | |
| | | | | AGTGA[CG]TGGACAAATTGCC | | |
| | | | | CATTCCAGAATACAATGGGATT | | |
| | | | | GAGAAATAATTGGG | | |
| VAMP8_P241_F | VAMP8 | NM_003761.2 | 14043025 | AAAAAAAGGCTGCCCTTTCTAG | hypomethylation | 21 |
| | | | | ATCAGGAGGTCCAGCCTCTGGA | | |
| | | | | AACCT[CG]GAGGGCTGCTTGA | | |
| | | | | TCTTTCTTTTCTAATTCCTGAC | | |
| | | | | AAGTTAGAAGACCT | | |
| ZAP70_P220_R | ZAP70 | NM_001079.3 | 46488942 | ACTGCTGCCTACCCTCCGGTTC | hypermethylation | 22 |
| | | | | CAGGTATGCAGGCTTCCTCCCT | | |
| | | | | TCTGA[CG]GTTCCTGCTGCTG | | |
| | | | | GAGTCGTCCTTCCTGAAACCCT | | |
| | | | | GCCTTTGCTTAGCC | | |
| IL1RN_P93_R | IL1RN | NM_173843.1 | 27894320 | GTCACCCTCCTGGAAACTGGGC | hypermethylation | 23 |
| | | | | CTGCTTGGCATCAAGTCAGCCA | | |
| | | | | TCAGC[CG]GCCCATCTCCTCA | | |
| | | | | TGCTGGCCAACCCTCTGTGAGT | | |
| | | | | GTGTGGGAGGGGAG | | |
| PADI4_P1011_R | PADI4 | NM_012387.1 | 6912575 | CCCAGGTGCAACCACAGCTCTG | hypermethylation | 24 |
| | | | | AGGCCACATGGGCATCCCCCTG | | |
| | | | | GCAGG[CG]TGGCCCACACCTG | | |
| | | | | CACTGTCTGGTCTGACACCCAG | | |
| | | | | AGGCCCTGGCAAGA | | |
| ERCC3_P1210_R | ERCC3 | NM_000122.1 | 4557562 | TCTTGAAGAGCCTTGGTAGAAG | hypomethylation | 25 |
| | | | | TATGGGCATTAAAGGTGATTCT | | |
| | | | | GGTGA[CG]GCTCAGATGGAAA | | |
| | | | | GGAGAAATATGTTATTGAAACT | | |
| | | | | GGAGGCAAGTGGTA | | |
| CASP10_P334_F | CASP10 | NM_001230.3 | 47078266 | TCGCTCCATTGTTTATTTGCAT | hypomethylation | 26 |
| | | | | GTGGACATAAGAAAGGGTTAAC | | |
| | | | | ATGGC[CG]ACAACTATTTCAT | | |
| | | | | GAGCTTTTTGGCTTTATTTGAA | | |
| | | | | AAGTGAAGTGTGTT | | |
| CTLA4_E176_R | CTLA4 | NM_005214.2 | 21361211 | AAGACCTGAACACCGCTCCCAT | hypermethylation | 27 |
| | | | | AAAGCCATGGCTTGCCTTGGAT | | |
| | | | | TTCAG[CG]GCACAAGGCTCAG | | |
| | | | | CTGAACCTGGCTACCAGGACCT | | |
| | | | | GGCCCTGCACTCTC | | |
| IGFBP5_P9_R | IGFBP5 | NM_000599.2 | 46094066 | TTCCTAGCTCTTTTCCCCTGCA | hypomethylation | 28 |
| | | | | GAAGTTTCCAAAGAGACTACGG | | |
| | | | | GGCTC[CG]GGAGAGCAGGCGC | | |
| | | | | TTTTAAATAGCCGGCCCCTGGC | | |
| | | | | TGCCAGCCAGTTTG | | |
| AGXT_P180_F | AGXT | NM_000030.1 | 4557288 | AAGAAACACTTCTCTCACCCCT | hypomethylation | 29 |
| | | | | GAGCTAAGCAGAATAAGAGGGG | | |
| | | | | CTGGA[CG]TGCAGGACTCAGA | | |
| | | | | GTGGGAGCGAGGAGGGCTGGGG | | |
| | | | | TGAGGACAGCTTTG | | |
| PTHR1_P258_F | PTHR1 | NM_000316.2 | 39995096 | TAAGAGAGAGGCATGGCAGGGC | hypermethylation | 30 |
| | | | | AAGGAGAGGACTATTGAGGCAC | | |
| | | | | ACACA[CG]TGTCTGGCAGCCT | | |
| | | | | GAGTGGGCCCAGTTACCTGGCA | | |
| | | | | GGCAGACCCATGGG | | |
| ZMYND10_P329__F | ZMYND10 | NM_015896.2 | 37594443 | CCCGCTGCTCTTCCTCCTCCTT | hypomethylation | 31 |
| | | | | ATGGCTTCTTGGTTCCTCTATT | | |
| | | | | TCTCG[CG]TCCCGGCTCCACT | | |
| | | | | AGTTGGCTCCTGAAATACTGCC | | |
| | | | | AGGGCGCACGACTT | | |
| IL17RB_E164_R | IL17RB | NM_018725.2 | 27477073 | CCAGCACCTCTTCCCTCATCTC | hypomethylation | 32 |
| | | | | CCGGCCCTCGAGCCCAGATCCT | | |
| | | | | GACGT[CG]TCTGATCCGCCAG | | |
| | | | | TCCAGGCTGCCCCGAAGGCGTG | | |
| | | | | CGCGGACTGCCGGC | | |
| CD86_P3_F | CD86 | NM_006889.2 | 29029570 | GAACAGCTTCTCTTAAAGAAAG | hypomethylation | 33 |
| | | | | TTAGCTGGGTAGGTATACAGTC | | |
| | | | | ATTGC[CG]AGGAAGGCTTGCA | | |
| | | | | CAGGGTGAAAGCTTTGCTTCTC | | |
| | | | | TGCTGCTGTAACAG | | |
| PADI4_E24_F | PADI4 | NM_012387.1 | 6912575 | AGGAACCAGCCCAGGGGCTTCC | hypomethylation | 34 |
| | | | | TACAGCCAGAGGGACGAGCTAG | | |
| | | | | CCCGA[CG]ATGGCCCAGGGGA | | |
| | | | | CATTGATCCGTGTGACCCCAGA | | |
| | | | | GCAGCCCACCCATG | | |
| RHOH_P953_R | RHOH | NM_004310.2 | 45827772 | GCCAACCTCTTTCCCACCTCAG | hypermethylation | 35 |
| | | | | GGCCTTTGCACATACTATTTGC | | |
| | | | | CTCTA[CG]TGGAATGTTCTTT | | |
| | | | | CCTCCTTCTCATCCATTAGAGT | | |
| | | | | GGCAGCAGTACTTT | | |
| FGF1_P357_R | FGF1 | NM_033136.1 | 15055540 | CAGGAACACAGAGCCATTGGCC | hypermethylation | 36 |
| | | | | AGCCAGGAGGGAGGTAGAGACA | | |
| | | | | GAAGA[CG]GTGGCAGCAGCTA | | |
| | | | | CCCTGGGTGTTATTTTAACGTG | | |
| | | | | GTTTGTCTTGGGGC | | |
| CSF1R_E26_F | CSF1R | NM_005211.2 | 27262658 | TCCTCTTCCTCTTCTCTCTTCT | hypomethylation | 37 |
| | | | | CCACCTTCTCCTCACTTCGTGC | | |
| | | | | TCTCA[CG]CTTTTGGACACTC | | |
| | | | | TGTCTGCCCTTCTCCTACCTGG | | |
| | | | | GGCCTGATCATGAC | | |
| SPARC_P195_F | SPARC | NM_003118.2 | 48675809 | GGTGGGCTGTCCTGACCAAACG | hypomethylation | 38 |
| | | | | TCCCAACCCTGCCTGCCTCATC | | |
| | | | | TGTTC[CG]GGGCTGCTGCCTA | | |
| | | | | AACCGACTCACAGAGTGCCAGG | | |
| | | | | GCTGGACAGGCCTG | | |
| ITK_P114_F | ITK | NM_005546.3 | 21614549 | TTTTTTACATATGCCTCCTCGT | hypermethylation | 39 |
| | | | | TTTGTGAATTTTGAAAGGATGT | | |
| | | | | GGTTT[CG]GCCTTTGACATCA | | |
| | | | | GAGGAGAAGCTCAGCTATGTTG | | |
| | | | | GCTGAACGTTGATA | | |
| ITK_E166_R | ITK | NM_005546.3 | 21614549 | CAAGAAATCCCAACAAAAGAGA | hypermethylation | 40 |
| | | | | AGAACTTCTCCCTCGAACTTTA | | |
| | | | | AAGTC[CG]CTTCTTTGTGTTA | | |
| | | | | ACCAAAGCCAGCCTGGCATACT | | |
| | | | | TTGAAGATCGTCAT | | |
| LTA_P214_R | LTA | NM_000595.2 | 6806892 | CTCCCAGCCCACGATTCCCCTG | hypermethylation | 41 |
| | | | | ACCCGACTCCCTTTCCCAGAAC | | |
| | | | | TCAGT[CG]CCTGAACCCCCAG | | |
| | | | | CCTGTGGTTCTCTCCTAGGCCT | | |
| | | | | CAGCCTTTCCTGCC | | |
| NOTCH4_E4_F | NOTCH4 | NM_004557.3 | 55770875 | TCTGCTCCCACTGCCCCTCTTC | hypomethylation | 42 |
| | | | | TTCCTCCTCGGCCTGCTGCAAG | | |
| | | | | CCTCA[CG]TCTGAGCTGTTTC | | |
| | | | | CTGAGTCACACAATGTCCTGGA | | |
| | | | | CACCCTAGTAATGG | | |
| NOTCH4_P938_F | NOTCH4 | NM_004557.3 | 55770875 | GTTGAGGCACTCATGGCTGCTG | hypermethylation | 43 |
| | | | | CTGGTGCACCTGAGAGCCTTCC | | |
| | | | | CCTAC[CG]GGGAATATACTTC | | |
| | | | | ACCAGCACCACTTTCTTCCTTT | | |
| | | | | TTTTAGCTTTTTAT | | |
| RUNX3_E27_R | RUNX3 | NM_001031680.1 | 72534651 | ATCATTAGATGGCGGGAAGGGG | hypermethylation | 44 |
| | | | | CTTTCGGCAGCCAGGGTGGAGG | | |
| | | | | AGCTC[CG]AAGCTGACAGAGC | | |
| | | | | AGAGTGGGCCGCCTCCAGTGCC | | |
| | | | | ACGGGGAATGAATG | | |
| GPR116_P850_F | GPR116 | NM_015234.3 | 44771172 | CCTCTGCAGCGCTCCCTTTCCC | hypermethylation | 45 |
| | | | | TTTCCCTTTCCTGGTTCTCAAG | | |
| | | | | GCTCC[CG]AGCTTATGCCTTT | | |
| | | | | TCTCCTTCTATGCTCCCATCCT | | |
| | | | | CATCATCCTGCAGC | | |
| RAB32_E314_R | RAB32 | NM_006834.2 | 20127508 | TGGTGATCGGCGAGCTTGGCGT | hypomethylation | 46 |
| | | | | GGGCAAGACCAGCATCATCAAG | | |
| | | | | CGCTA[CG]TCCACCAGCTCTT | | |
| | | | | CTCCCAGCACTACCGGGCCACC | | |
| | | | | ATCGGGGTGGACTT | | |
| ESR1_P151_R | ESR1 | NM_000125.2 | 62821793 | GGCACATAAGGCAGCACATTAG | hypomethylation | 47 |
| | | | | AGAAAGCCGGCCCCTGGATCCG | | |
| | | | | TCTTT[CG]CGTTTATTTTAAG | | |
| | | | | CCCAGTCTTCCCTGGGCCACCT | | |
| | | | | TTAGCAGATCCTCG | | |
| IL6_P213_R | IL6 | NM_000600.1 | 10834983 | AAGAAAGTAAAGGAAGAGTGGT | hypomethylation | 48 |
| | | | | TCTGCTTCTTAGCGCTAGCCTC | | |
| | | | | AATGA[CG]ACCTAAGCTGCAC | | |
| | | | | TTTTCCCCCTAGTTGTGTCTTG | | |
| | | | | CCATGCTAAAGGAC | | |
| CLDN4_P1120_R | CLDN4 | NM_001305.3 | 34335232 | CTCCCCAGCCCAGTCTCTGGTC | hypermethylation | 49 |
| | | | | AAACTGGATTCCTGGCTGTTCC | | |
| | | | | CAGAA[CG]AGCTGCCTTTCCC | | |
| | | | | CACCTTGCCACCTCTGCCCTTG | | |
| | | | | TTCTCTCTGCCTGA | | |
| HGF_E102_R | HGF | NM_001010933.1 | 58533164 | GGGCTGGCGGATCCCTCTGGAG | hypomethylation | 50 |
| | | | | GAGATGCCTGGGTGAAAGAATC | | |
| | | | | CTGTT[CG]GAGTCAGTGCCTA | | |
| | | | | AAAGAGCCAGTCGGCTCTGAGC | | |
| | | | | TGCTTTTTATTGCG | | |
| TFPI2_P152_R | TFPI2 | NM_006528.2 | 31543803 | ACCCCGCCGCCCCCGCGCTGCA | hypomethylation | 51 |
| | | | | AACTGTGTAAGAGGGAGAGGAA | | |
| | | | | TTCCC[CG]CCAAGTTGAAAAG | | |
| | | | | TTGAACCTGCCTCCCAAACTTT | | |
| | | | | CTCCTGTAGTCCAG | | |
| TRIP6_P1274_R | TRIP6 | NM_003302.1 | 23308730 | TCCTGCTGCAGATGGCAACCAT | hypomethylation | 52 |
| | | | | CTTGGGCATGGTGCCCGCTTGG | | |
| | | | | CATAG[CG]CCCGGCTCCGGAT | | |
| | | | | CTTCCTGTGCCTGGGGCCTCGG | | |
| | | | | GAGGCGCCTGGGGC | | |
| RUNX3_P247_F | RUNX3 | NM_001031680.1 | 72534651 | CACAGGATGCGAGAAGCCTGCT | hypermethylation | 53 |
| | | | | CGCGGCCTTGGCTCATTGGCTG | | |
| | | | | GGCCG[CG]GTCACCTGGGCCG | | |
| | | | | TGATGTCACGGCCTTTTAGAAG | | |
| | | | | ATCTTGTGGCTGCC | | |
| TRIP6_P1090_F | TRIP6 | NM_003302.1 | 23308730 | GGCTGGGGAACCCGAGGCGGAG | hypomethylation | 54 |
| | | | | GAGGAAGGGGACTTTGTGAACA | | |
| | | | | GTGGG[CG]GGGAGACGCAGAG | | |
| | | | | GCAGAGGCCCTGGCACGCAGCG | | |
| | | | | CCAACGCCCTGGTT | | |
| CPA4_E20_F | CPA4 | NM_016352.2 | 61743915 | AGACTCTTTATAAATACAGCTT | hypermethylation | 55 |
| | | | | GACTCAGCCACTGTATGACTGA | | |
| | | | | CTCCC[CG]GGGACATGAGGTG | | |
| | | | | GATACTGTTCATTGGGGCCCTT | | |
| | | | | ATTGGGTCCAGCAT | | |
| SYK_P584_F | SYK | NM_003177.3 | 34147655 | CCATTCTTAGGGCTATAGGTTT | hypomethylation | 56 |
| | | | | AATTTATTTGGTTGTGGACGTC | | |
| | | | | AGAGC[CG]TCATGGTAAGAAG | | |
| | | | | GAAGCAAAGCCTTTTGTAATAA | | |
| | | | | TTAAAGCCTTCAGA | | |
| LCN2_P141_R | LCN2 | NM_005564.2 | 38455401 | GTTGTCCCTGCCAGAGGTGCAG | hypomethylation | 57 |
| | | | | CACTCCGGGAATGTCCCTCACT | | |
| | | | | CTCCC[CG]TCCCTCTGTCTTG | | |
| | | | | CCCAATCCTGACCAGGTGCAGA | | |
| | | | | AATCTTGCCAAGTG | | |
| LCN2_P86_R | LCN2 | NM_005564.2 | 38455401 | TCTGTCTTGCCCAATCCTGACC | hypomethylation | 58 |
| | | | | AGGTGCAGAAATCTTGCCAAGT | | |
| | | | | GTTTC[CG]CAGGAGTTGCTGG | | |
| | | | | CAATTGCCTCACATTCCTGGCC | | |
| | | | | TTGGCAAAGAATGA | | |
| SLC22A18_P472__R | SLC22A18 | NM_002555.3 | 34734074 | TGCCCAGCGCTCCCAGGGTCAC | hypomethylation | 59 |
| | | | | CCCTCTCTCTAGACTCACTTTC | | |
| | | | | TGCCC[CG]TCACCCCACTGTA | | |
| | | | | CACCCTTGGTCCCAGCCCCTTC | | |
| | | | | CAGTGGCTCAGCTT | | |
| SLC22A18P216_R | SLC22A18 | NM_002555.3 | 34734074 | AGATGAGCCAAAGCCCTTCCTT | hypomethylation | 60 |
| | | | | CCTCCAGTCAGCCTGGATCCTC | | |
| | | | | TCATC[CG]GCAGAACTGTCGC | | |
| | | | | CTTGCTTCTCTGAAGCGGTGAA | | |
| | | | | TGCCCTGGGGCTGG | | |
| RUNX3_P393_R | RUNX3 | NM_001031680.1 | 72534651 | GAGAAATAGAAAAGTGATGGCT | hypermethylation | 61 |
| | | | | TTTATTTGTGAGGCTGGCCTCA | | |
| | | | | GCACG[CG]GCCCAAGAAACAG | | |
| | | | | AACTGAAAGCGGTTGCAGTGGG | | |
| | | | | CGTGGCCAGGAGGG | | |
| LMO2_E148_F | LMO2 | NM_005574.2 | 6633806 | TTGGTGGCCTGGTTGTCTATCT | hypomethylation | 62 |
| | | | | GATAGGGCGGAGCCTTCACCCT | | |
| | | | | TGCAG[CG]AGCTCTCTCACAC | | |
| | | | | CAGATGTGCTCTGCGTGGAATC | | |
| | | | | CTAGGCCATCAGGG | | |
| LMO2_P794_R | LMO2 | NM_005574.2 | 6633806 | CAGCTACCTCCCCCGCATGCAT | hypomethylation | 63 |
| | | | | GTCTGTCTGCTGGGCAAGGCCC | | |
| | | | | AATTC[CG]AGGTGACAGCTCA | | |
| | | | | CCGGGCCTCACCCACAAGTCTC | | |
| | | | | TTCCAAGCATTAGC | | |
| CD82_P557_R | CD82 | NM_002231.3 | 67782352 | GATTCAATCAATGGTAGTCAGT | hypomethylation | 64 |
| | | | | ATTTTCAAAAAGTTCCTGGGCC | | |
| | | | | CAGGC[CG]CCTCCTGATAGAG | | |
| | | | | GCCCCGACTTAGGACACAAACC | | |
| | | | | GCTCCCACGCCGTT | | |
| SPI1_E205_F | SPI1 | NM_003120.1 | 4507174 | GAGTCCCGGTACTCACAGGGGG | hypomethylation | 65 |
| | | | | GACGAGGGGAAACCCTTCCATT | | |
| | | | | TTGCA[CG]CCTGTAACATCCA | | |
| | | | | GCCGGGCTCCGAGTCGGTCAGA | | |
| | | | | TCCCCTGCCTCGGT | | |
| SPI1_P48_F | SPI1 | NM_003120.1 | 4507174 | TTATCGAAGGGCCTGCCGCTGA | hypomethylation | 66 |
| | | | | GGAGATAGTCCCCTTGGGGTGC | | |
| | | | | ATCAC[CG]CCCCAACCCGTTT | | |
| | | | | GCATAAATCTCTTGCGCTACAT | | |
| | | | | ACAGGAAGTCTCTG | | |
| KCNK4_E3_F | KCNK4 | NM_016611.2 | 15718764 | CCGATCCGGTAATGGGCCTGGG | hypomethylation | 67 |
| | | | | AGATGCCAGATTAGCGTGGTGC | | |
| | | | | CTGTC[CG]GAGAGACGGGCCA | | |
| | | | | GCTGATGCCCAGGTCGGGGCCC | | |
| | | | | TGCCGCTGGCCACA | | |
| MMP8_E89_R | MMP8 | NM_002424.1 | 4505220 | CAGGAAAGGCCTTGGAAATCTG | hypomethylation | 68 |
| | | | | CACATGGAGTAAGAGCAGAAAT | | |
| | | | | GGAAG[CG]TCTTCAGGGAGAA | | |
| | | | | CATGATCTTCTCTTCAAACTCT | | |
| | | | | ACCCCTCCTGGCTT | | |
| CD9_P585_R | CD9 | NM_001769.2 | 21237762 | TTTGCTAATTACTTCCAAAAGC | hypomethylation | 69 |
| | | | | CTCCCATCTGTCATCCCACCCA | | |
| | | | | GACTG[CG]CGCTTCTAATTCC | | |
| | | | | TCCTACCCCACATGCTGTGCCC | | |
| | | | | AATGAAAAGTATGG | | |
| CD9_P504_F | CD9 | NM_001769.2 | 21237762 | TGCCCAATGAAAAGTATGGTCA | hypomethylation | 70 |
| | | | | GCGAGCGAAGGTTTGCAAGGAG | | |
| | | | | ACAGA[CG]AGGGCGAAATTAA | | |
| | | | | GCCAGGCGGCTTCCCTTTAAAT | | |
| | | | | CCTCGCAAAGCAGA | | |
| LCK_E28_F | LCK | NM_005356.2 | 20428651 | GCAGCCAGGTTAGGCCAGGAGG | hypermethylation | 71 |
| | | | | ACCATGTGAATGGGGCCAGAGG | | |
| | | | | GCTCC[CG]GGCTGGGCAGGTA | | |
| | | | | AGGAGCGCTGGTATTGGGGGCG | | |
| | | | | CAGGCGCCGGGGTG | | |
| TNFRSF1A_P678__F | TNFRSF1A | NM_001065.2 | 23312372 | GTCCCCCCACCCTGCCCCACTG | hypomethylation | 72 |
| | | | | TTGATCCTGGCTCTGCCACCAA | | |
| | | | | TCATG[CG]ACATCAGGCAACT | | |
| | | | | CCTCTCCTAAGCCTCTGTTGGT | | |
| | | | | TCCTTGTTTATTAA | | |
| PTPN6_P282_R | PTPN6 | NM_080548.2 | 34328901 | AGGAACTGGGCTGTTAGGGATT | hypomethylation | 73 |
| | | | | TTCCTTAGGCCCTTTGGTTTCC | | |
| | | | | GCCTA[CG]GAGAGGTTTCCCC | | |
| | | | | CATTGGTTGCTCTTCCTCAGCC | | |
| | | | | AGGGTTACTTCCTG | | |
| TM7SF3_P1068_R | TM7SF3 | NM_016551.1 | 7706574 | ACCACTGCAACTGGGTCTTGCA | hypomethylation | 74 |
| | | | | GTGGGGAAGAGGGACTGGGCTC | | |
| | | | | AACTC[CG]AATACAGCGTGGG | | |
| | | | | CAAGAGGGAATTTATAGCCAAC | | |
| | | | | CAGCAGTATGGAGT | | |
| KRT1_P798_R | KRT1 | NM_006121.2 | 17318568 | GGATAGCATGCAAACGCCCTTG | hypermethylation | 75 |
| | | | | AGTGAAAAAGCCCACAGAGCAG | | |
| | | | | TGAGA[CG]AGTAAATAGAAGC | | |
| | | | | TCTAGGACATTTTGTAAAGCAC | | |
| | | | | AGGGGTGGAGGTGA | | |
| IFNG E293_F | IFNG | NM_000619.2 | 56786137 | AATGACTGCCTACAAGAGATGA | hypermethylation | 76 |
| | | | | CAGCCTATCAGAGATGCTACAG | | |
| | | | | CAAGT[CG]ATATTCAGTCATT | | |
| | | | | TTCAACCACAAACAAGTACTAT | | |
| | | | | TAAAAAGTCATACT | | |
| IFNG_P459_R | IFNG | NM_000619.2 | 56786137 | AGCCTTTTAAAATTTTTCTTGC | hypermethylation | 77 |
| | | | | AAATGACCAGAAAGCAAGGAAA | | |
| | | | | GAATG[CG]GTTAAAAGAACAA | | |
| | | | | TTTGGTGAGGAAGTCCTTCATC | | |
| | | | | AGAGTTGGTTAGTA | | |
| MMP14_P13_F | MMP14 | NM_004995.2 | 13027797 | CGGGGACGGAGGAGAGGCTGTG | hypomethylation | 78 |
| | | | | GGAGAAGGGAGGGACCAGAGGA | | |
| | | | | GAGAG[CG]AGAGAGGGAACCA | | |
| | | | | GACCCCAGTTCGCCGACTAAGC | | |
| | | | | AGAAGAAAGATCAA | | |
| BCL2L2_P280_F | BCL2L2 | NM_004050.2 | 14574571 | CCAGGCACACAGTTCAGGGCTG | hypomethylation | 79 |
| | | | | GAAAAGTTCAACAAGTGCATGG | | |
| | | | | AACAT[CG]GAAACCTCCTGAA | | |
| | | | | AATGCTAAATTTGCCCCGAGAT | | |
| | | | | GTCCCGAAGTCCGG | | |
| CRIP1_P874_R | CRIP1 | NM_001311.3 | 39725694 | GCCTGGCACCGGGACCATCCTC | hypomethylation | 80 |
| | | | | CGCCTCAACTTTGCAGCGTACT | | |
| | | | | TGGAC[CG]CTCTGGCCGCCCT | | |
| | | | | GGGCGCTACCCGCAGAGATAAG | | |
| | | | | GGCCCCTCCCTGCG | | |
| APBA2_P227_F | APBA2 | NM_005503.2 | 22035549 | CCTTTGGAAATAAACACGAAGG | hypermethylation | 81 |
| | | | | TTCACTTGAAGACTTGGGGGAG | | |
| | | | | AATCA[CG]GTCAACTTGTGAC | | |
| | | | | GCTTGGTTTTTCAGATATTCAG | | |
| | | | | CTGCTCTGGAGAGC | | |
| CSF3R_P8_F | CSF3R | NM_172313.1 | 27437044 | AGAAGTTCCTGAAACCAGCTGC | hypomethylation | 82 |
| | | | | AGTCCAGCTTCTCTCCCCGAGC | | |
| | | | | TCTGT[CG]TTAATGGCTCAGC | | |
| | | | | CTCTGACAGGCCCGGGGGCTGG | | |
| | | | | GGATTGCAACACCT | | |
| CARD15_P302_R | CARD15 | NM_022162.1 | 11545911 | TGGTGATGTAGCTGCTGGGAGG | hypomethylation | 83 |
| | | | | ACAGAGCTCCGAGTCACGTGGC | | |
| | | | | TTGGG[CG]GGCCTCCCCTTCC | | |
| | | | | TGGTGTCCACAGAAGCCCAACG | | |
| | | | | TCACTAGCTGGGGT | | |
| ALOX12_E85_R | ALOX12 | NM_000697.1 | 4502050 | GGCCGCTACCGCATCCGCGTGG | hypomethylation | 84 |
| | | | | CCACCGGGGCCTGGCTCTTCTC | | |
| | | | | CGGGT[CG]TACAACCGCGTGC | | |
| | | | | AGCTTTGGCTGGTCGGGACGCG | | |
| | | | | CGGGGAGGCGGAGC | | |
| MFAP4_P197_F | MFAP4 | NM_002404.1 | 23111004 | GGGAGGTGGGGCTGGAGCCAGG | hypomethylation | 85 |
| | | | | GGACCACCTGTGTCTCATTAGT | | |
| | | | | CCTGT[CG]GGCAAAGTACTGC | | |
| | | | | AGACGTTAACTCCCTGCTGGCT | | |
| | | | | CCAACTGTTCCCTG | | |
| GRB7_P160_R | GRB7 | NM_001030002.1 | 71979666 | CGGGACTCTTGATCTTCGCTCG | hypomethylation | 86 |
| | | | | TGGTACTGTCTGTTCGGCTGTC | | |
| | | | | TTCCC[CG]CCTCTCCCCAGGC | | |
| | | | | ACCTGCATCCTCCCTTGGCACC | | |
| | | | | TGCTGCCAGGCTAG | | |
| GRB7_E71_R | GRB7 | NM_001030002.1 | 71979666 | ATCTGGACACACAGGGCTCCCC | hypomethylation | 87 |
| | | | | CCCGCCTCTGACTTCTCTGTCC | | |
| | | | | GAAGT[CG]GGACACCCTCCTA | | |
| | | | | CCACCTGTAGAGAAGCGGGAGT | | |
| | | | | GGATCTGAAATAAA | | |
| CSF3_E242_R | CSF3 | NM_172220.1 | 27437050 | TGTCCCCGAGAGGGCCTCAGGT | hypomethylation | 88 |
| | | | | GGTAGGGAACAGCATGTCTCCT | | |
| | | | | GAGCC[CG]CTCTGTCCCCAGC | | |
| | | | | CCTGCAGCTGCTGCTGTGGCAC | | |
| | | | | AGTGCACTCTGGAC | | |
| RARA_P1076_R | RARA | NM_000964.2 | 75812906 | GTCTTCTCCCCTTCTAGGGAGA | hypomethylation | 89 |
| | | | | GGCCATGCCCTCTCCCCTCAAG | | |
| | | | | TCTGT[CG]CTGACTTCCTCTG | | |
| | | | | GCCCTTCCCCTCATGACGTTTT | | |
| | | | | CCCTGCTCTGCTGC | | |
| STAT5A _P704_R | STAT5A | NM_003152.2 | 21618341 | ACCCAAATGTGGCAATGGGTTT | hypomethylation | 90 |
| | | | | GTATCCAGCCACCGACAGGCTG | | |
| | | | | CATGA[CG]GTGGCAAAGTCAC | | |
| | | | | TTCCCCTCTCTGGCCTTTGTTT | | |
| | | | | TTCCACTTGTAAAA | | |
| CSF3R_P472_F | CSF3R | NM_172313.1 | 27437044 | GGTTCCAGGGAATTGTGTAACC | hypomethylation | 91 |
| | | | | CAATACTCACTGCTCCCCTCTT | | |
| | | | | CATTA[CG]TATTCTGTGCATT | | |
| | | | | GCCCATAGACCAGGCAGATGGA | | |
| | | | | GAAACAGGAATTCT | | |
| PECAM1__E32_R | PECAM1 | NM_000442.2 | 21314616 | AAATTGCTCTGGTCACTTCTCC | hypomethylation | 92 |
| | | | | CGGCGCCTGCAGAGAGACCGGC | | |
| | | | | TGTGG[CG]CTGGTCAGGTAAT | | |
| | | | | GGCAGCCATGGCTGGAAACCGG | | |
| | | | | GAACAATGGGGCCT | | |
| PECAM1_P135_F | PECAM1 | NM_000442.2 | 21314616 | GTTTAGTTTCTTTAGGGAAAAA | hypomethylation | 93 |
| | | | | ACAAGGCACAAGTGACATTTGC | | |
| | | | | CTTGG[CG]TTCTTGACCCTCC | | |
| | | | | CTCTGTCTCGCCTGGGTTTGGG | | |
| | | | | GGCCCTTCTCATGG | | |
| SEPT9_P374_F | SEPT9. | NM_006640.2 | 19923366 | TGGGGTACAGGGTGAAGAAGGG | hypomethylation | 94 |
| | | | | CTGGGGCCAGCCCAGGACAGAG | | |
| | | | | GAAGG[CG]AGGCAGGCACGCA | | |
| | | | | GGAACTGGGCTTTTTAAACCCT | | |
| | | | | TAAGCCCAAGGAAA | | |
| MATK_P190_R | MATK | NM_139355.1 | 21450845 | GGGTGGGAGGCTTCCGAGAGCC | hypomethylation | 95 |
| | | | | GCCTCTCCCGGGGCATAAGGAA | | |
| | | | | GGAAG[CG]GGGCTGCAGGTAC | | |
| | | | | CGCCTGGGGTTCACAGCAGGGG | | |
| | | | | ACGAGGTGCCTCCC | | |
| EMR3_E61_F | EMR3 | NM_152939.1 | 23397638 | AGCTGACTCATGAAATTGCTAT | hypomethylation | 96 |
| | | | | CAGAAAAGCAAACTGCTTCCCC | | |
| | | | | TCTTT[CG]CCATCAGACTCAT | | |
| | | | | GGTTCTGCTTTTCGTTTATTTG | | |
| | | | | CTGTACCTTTTCTG | | |

Any appropriate method can be used to obtain a blood sample that can be processed to obtain nucleic acid for the assessment of the human's CpG methylation site profile. For example, leukocyte nucleic acid can be obtained and assessed as described herein to determine whether any one or more of the methylation CpG sites listed in Table 1 or 5 have an altered level of methylation as compared to controls (e.g., healthy humans known to not have pancreatic cancer). In some cases, combinations of methylation CpG sites can be assessed as described herein. Examples of such combinations include, without limitation, (a) IL10_P348, LCN2_P86, ZAP70_P220, AIM2_P624, and TAL1_P817; (b) LCN2_P86, ZAP70_P220, AIM2_P624, and TAL1_P817; (c) IL10_P348, ZAP70_P220, AIM2_P624, and TAL1_P817; (d) IL10_P348, LCN2_P86, AIM2_P624, and TAL1_P817; (e) IL10_P348, LCN2_P86, ZAP70_P220, and TAL1_P817; (f) IL10_P348, LCN2_P86, ZAP70_P220, and AIM2_P624; (g) IL10_P348, LCN2_P86, and ZAP70_P220; (h) IL10_P348, LCN2_P86, and AIM2_P624; (i) IL10_P348, LCN2_P86, and TAL1_P817; (j) IL10_P348, ZAP70_P220, and AIM2_P624; (k) IL10_P348, ZAP70_P220, and TAL1_P817; (l) IL10_P348, AIM2_P624, and TAL1_P817; (m) LCN2_P86, ZAP70_P220, and AIM2_P624; (n) LCN2_P86, ZAP70_P220, and TAL1_P817; (o) LCN2_P86, AIM2_P624, and TAL1_P817; (p) ZAP70_P220, AIM2_P624, and TAL1_P817; (q) IL10_P348 and LCN2_P86; (r) IL10_P348 and ZAP70_P220; (s) IL10_P348 and AIM2_P624; (t) IL10_P348 and TAL1_P817; (u) LCN2_P86 and ZAP70_P220; (v) LCN2_P86 and AIM2_P624; (w) LCN2_P86 and TAL1_P817; (x) ZAP70_P220 and AIM2_P624; (y) ZAP70_P220 and TAL1_P817; and (z) AIM2_P624 and TAL1_P817.

Any appropriate method can be used to assess a methylation CpG site for methylation level change (e.g., the presence or absence of a methyl group). For example, methylation assays available commercially (e.g., from Illumina) can be used to determine the methylation state of methylation CpG sites.

Once a human is determined to having altered levels of methylation of methylation CpG sites that are indicative of pancreatic cancer, then the human can be classified as having pancreatic cancer or can be evaluated further to confirm a diagnosis of pancreatic cancer. Humans identified as having pancreatic cancer as described herein can be treated with any appropriate pancreatic cancer treatment including, without limitation, surgery, radiation, and chemotherapy.

The invention will be further described in the following examples, which do not limit the scope of the invention described in the claims.

### EXAMPLES

### Example 1

### Leukocyte DNA Methylation Signature Differentiates Pancreatic Cancer Patients from Healthy Controls

### Study Population

PaC index cases were adult patients with a histologically confirmed primary adenocarcinoma of the pancreas seen at Mayo Clinic. Eligible Mayo pancreatic adenocarcinoma cases were identified through an ultra-rapid patient identification system and recruited into a prospective research registry. Study coordinators identified potential patients from the electronic patient scheduling system and daily pathology reports. All eligible patients were contacted either in the clinic at the time of their appointment, or later by mail or phone if clinic contact was not possible. If contacted at the clinic, a study coordinator obtained informed consent, arranged a venipuncture for 40 mL of blood prior to start of treatment (whenever possible), and asked the participant to complete the study questionnaire. If mail contact was required (approximately 28% of the cases were approached by mail), the study coordinator mailed an invitation letter to the patient's home address. A follow-up telephone call was made if the sample or forms were not received after one month. About 74% of all eligible patients were enrolled into the registry. From the registry, 132 never-smoker patients in phase I and 240 patients in phase II were selected with equal representation of sex, smoking status (smoker/nonsmoker) and stage of PaC (resectable, locally advanced and metastatic).

The healthy Caucasian controls were selected from a Mayo Clinic—based research registry of primary care control patients having routine check-up visits (general medical exam). Controls were frequency-matched to cases on age (±5 years), sex, and state/region of residence distribution of the cases. Controls had no previous diagnosis of cancer (except non-melanoma skin cancer) at the time of enrollment. Prior to their appointment, potential controls were mailed an information brochure describing the study and a letter of invitation. On the day of the appointment, a study assistant approached the subject, confirmed eligibility criteria, and obtained informed consent. Each participant completed study questionnaires (which included a self-report of height, weight, and diabetes status) and provided 30 mL of research blood sample. About 70% of all approached controls participated in this study. From this registry, 60 never smoker controls for phase I and 240 controls (half are never smokers) for phase II were selected.

### DNA Modification by Sodium Bisulfite

DNA was extracted from 5 mL of whole blood utilizing an AutoGen FlexStar (AutoGen, Inc., Mass.), and the genomic DNA specimens were modified using the EZ DNA Methylation kit from Zymo Research Corporation (Orange, Calif.) that combined bisulfite conversion and DNA cleaning. The kit is based on the three-step reaction that takes place between cytosine and sodium bisulfite where cytosine is converted into uracil. 1 μg of genomic DNA from peripheral blood DNA was used for the modification per manufacturer recommendation. Treated DNA specimens were stored at −20° C. and were assayed within two weeks.

### DNA Methylation Profiling Analysis

The Illumina (San Diego, Calif.) GoldenGate methylation Beadchip (cancer panel) and Illumina custom VeraCode methylation assay were used for phase I and phase II, respectively, following the manufacturer's procedure. The arrays were imaged using a BeadArray Reader scanner (Illumina, Inc.). The proportion methylated (β-value) at each CpG site was calculated using BeadStudio Software (Illumina, Inc.) after subtracting background intensity, which was computed from negative controls, from each analytical data point. The β-value represented relative ratio of fluorescent signals between the M (methylated) allele and M+U (unmethylated) alleles. This value ranges continuously from 0 (unmethylated) to 1 (fully methylated).

### Differential Methylation Analysis

Due to non-Gaussian distribution of the CpG methylation values, Wilcoxon Rank Sum tests were used to examine differences in median β-values between cases and controls in both phase I and phase II. To correct for multiple testing in phase I, q-values were used to represent the false discovery rate (FDR) (Storey and Tibshirani, Proc. Natl. Acad. Sci. USA, 100(16):9440-5 (2003)). The CpGs with a FDR q-value≦0.05 level were considered significant. These CpGs were then candidates for phase II validation, where a p-value≦0.05 was considered significant. Bland-Altman plots were used to evaluate agreement between the two methylation assays in the 40 subjects assayed in both phase I and phase II. These plots allow evaluation of assay disagreement as a function of level of methylation (Bland and Altman, Lancet, 1(8476):307-10 (1986)).

### Prediction Model Building

To develop prediction models, likelihood cross-validated penalized logistic regression models, which implemented either an L1 penalty (Lasso) (Tibshirani, J. Royal Statist. Soc. B, 58(1):267-88 (1996)) or an L2 penalty (Ridge) using the R package ‘penalized,’ were used (Goeman, Biometrical Journal, 52(1):70-84 (2010)). A Lasso model (or L1 penalty) was utilized in phase I testing study because of its desirable feature for model selection, which has a minimal effect on associated CpG coefficients while setting the unassociated CpGs' coefficients to zero. A Ridge regression model (or L2 penalty) that shrinks all coefficients to small values but not zeros was also considered for model building. The variable selection process is governed by a parameter that forces all coefficients to be shrunk near zero initially, then is gradually released to reduce the amount of shrinkage. The optimal value of this parameter is determined via cross validation. The Ridge model results were also compared to results from the Lasso model to hone the final model.

The final model identified through the penalized approaches was then fit as a generalized linear model (logistic regression) using the R package ‘glm’, in order to estimate the area under (AUC) the receiver operating characteristic (ROC) curve for each model. Models were fitted in both the testing set (phase I) and the validation set (phase II) separately with AUC reported for each model. In addition to the unadjusted model (only the CpGs), two more models were fitted, one that considered age, sex, and first degree family history as covariates and another that also considered ABO blood type (‘O’ vs ‘non-O’) as an additional covariate. ABO blood types were derived for a subset of patients which had GWAS genotype information (Petersen et al., Nat. Genet., 42(3):224-8 (2010)) available. The phase II models were fit two ways. First, coefficients from phase I were held fixed and discrimination assessed. Second, since the assay platform changed from phase I to phase II, the models were fit allowing the coefficients to be re-estimated.

### Identification of Differentially Methylated CpG Sites in Phase I

For phase I, 132 never-smoker patients with PaC and 60 never-smoker healthy controls were examined. Due to chemo- or radiation therapy before blood was drawn, 13 patients were excluded from this analysis. The methylation status (β values) of 1,505 CpG sites from leukocyte DNAs in the remaining 119 cases and 60 controls were evaluated (Table 2). Because significant methylation differences on the X chromosome exist between males and females, CpG sites on autosomes and sex chromosome were analyzed separately. These analyses identified significant differences at 110 CpG sites in 92 independent genes (FDR≦0.05). 109 of the 110 significant CpG sites were located on autosomes. Table 3 lists the 10 most significant CpG sites in the phase I study.

TABLE 2
Subject demographics for Phases I and II.

| | Phase I | | | | | Phase II | | | | |
|---|---|---|---|---|---|---|---|---|---|---|
| | Controls | | Cases | | P- | Controls | | Cases | | P- |
| Variable | (N = 60) | | (N = 119) | | value | (N = 215) | | (N = 173) | | value |
| Age | | | | | 1.00 | | | | | 1.00 |
| ≦49 | 3 | (5%) | 5 | (4%) | | 20 | (9%) | 15 | (9%) | |
| 50-54 | 4 | (7%) | 8 | (7%) | | 14 | (7%) | 10 | (6%) | |
| 55-59 | 7 | (12%) | 12 | (10%) | | 28 | (13%) | 21 | (12%) | |
| 60-64 | 7 | (12%) | 12 | (10%) | | 33 | (15%) | 26 | (15%) | |
| 65-69 | 12 | (20%) | 25 | (21%) | | 39 | (18%) | 33 | (19%) | |
| 70-74 | 11 | (18%) | 22 | (18%) | | 32 | (15%) | 22 | (13%) | |
| 75-79 | 11 | (18%) | 22 | (18%) | | 29 | (13%) | 29 | (17%) | |
| 80-84 | 3 | (5%) | 8 | (7%) | | 16 | (7%) | 14 | (8%) | |
| ≧85 | 2 | (3%) | 5 | (4%) | | 4 | (2%) | 3 | (2%) | |
| Sex | | | | | 0.87 | | | | | 0.90 |
| Female | 31 | (52%) | 60 | (50%) | | 108 | (50%) | 88 | (51%) | |
| Male | 29 | (48%) | 59 | (50%) | | 107 | (50%) | 85 | (49%) | |
| Family History of | | | | | | | | | | |
| Pancreas Cancer | | | | | | | | | | |
| (1^{st} degree) | | | | | | | | | | |
| No | 58 | (97%) | 104 | (87%) | 0.046 | 196 | (91%) | 147 | (85%) | 0.06 |
| Yes | 2 | (3%) | 15 | (13%) | | 19 | (9%) | 26 | (15%) | |
| Smoking Status | | | | | | | | | | 0.90 |
| Never Smokers | 60 | (100%) | 119 | (100%) | | 97 | (45%) | 77 | (45%) | |
| Ever Smokers | 0 | — | 0 | — | | 118 | (55%) | 96 | (55%) | |
| Stage of Pancreas | | | | | | | | | | |
| Cancer | | | | | | | | | | |
| Resectable | | | 31 | (26%) | | | | 58 | (34%) | |
| Locally | | | 45 | (38%) | | | | 59 | (34%) | |
| Advanced | | | | | | | | | | |
| Metastatic | | | 43 | (36%) | | | | 56 | (32%) | |
| GWAS | | | | | <0.001 | | | | | 0.028 |
| genotyping | | | | | | | | | | |
| No | 34 | (57%) | 32 | (27%) | | 106 | (49%) | 66 | (38%) | |
| Yes | 26 | (43%) | 87 | (73%) | | 109 | (51%) | 107 | (62%) | |

TABLE 3
Top 10 most differentially methylated CpG sites
in phase I and validation in phase II.

| | Median β | Median | Difference | |
|---|---|---|---|---|
| Illumina ID | Control | β Case | (case-control) | p value |
| | Phase I | | | |
| ITK_P114_F | 0.8337 | 0.9006 | 0.0669 | <1E−10 |
| LCN2_P86_R | 0.5608 | 0.4398 | −0.121 | 2.00E−10 |
| ITK_E166_R | 0.8859 | 0.9414 | 0.0555 | 5.00E−10 |
| PECAM1_E32_R | 0.2319 | 0.1566 | −0.0753 | 1.60E−09 |
| LMO2_E148_F | 0.3885 | 0.2704 | −0.1181 | 2.30E−09 |
| IL10_P348_F | 0.6026 | 0.4597 | −0.1429 | 2.50E−09 |
| LCK_E28_F | 0.8114 | 0.8684 | 0.057 | 3.60E−09 |
| RUNX3_P247_F | 0.7837 | 0.8672 | 0.0835 | 5.90E−09 |
| LMO2_P794_R | 0.3143 | 0.2027 | −0.1116 | 1.02E−08 |
| MMP14_P13_F | 0.4721 | 0.3472 | −0.1249 | 2.27E−08 |
| | Phase II | | | |
| ITK_P114_F | 0.846 | 0.8898 | 0.0438 | <1E−10 |
| LCN2_P86_R | 0.591 | 0.4993 | −0.0917 | <1E−10 |
| ITK_E166_R | 0.8885 | 0.9299 | 0.0414 | <1E−10 |
| PECAM1_E32_R | 0.2851 | 0.2211 | −0.064 | <1E−10 |
| LMO2_E148_F | 0.4969 | 0.3904 | −0.1065 | <1E−10 |
| IL10_P348_F | 0.7191 | 0.6382 | −0.0809 | <1E−10 |
| LCK_E28_F | 0.8593 | 0.8999 | 0.0406 | <1E−10 |
| RUNX3_P247_F | 0.7528 | 0.841 | 0.0882 | <1E−10 |
| LMO2_P794_R | 0.3754 | 0.3027 | −0.0727 | 6.00E−10 |
| MMP14_P13_F | 0.5694 | 0.4807 | −0.0887 | <1E−10 |

To evaluate possible methylation changes during tumor progression, the methylation differences among three stages of PaC within this patient population, including 31 resectable, 45 locally advanced, and 43 metastatic cases, were examined. Although nine CpG sites showed a trend in association with clinical stages (p<0.01) (Table 4), the data analysis did not reveal significant difference among the three stages (all CpG sites with FDR>0.05).

TABLE 4
Top 10 most differentially methylated CpG sites among 3 clinical stages.

| | | Mean β values | | | | |
|---|---|---|---|---|---|---|
| | Gene | | Locally | | p | |
| Illumina ID | Name | Resectable | Advanced | Metastatic | value | FDR |
| ZMYND10_P329_F | ZMYND10 | 0.045 | 0.032 | 0.019 | 0.001 | 0.722 |
| EPO_P162_R | EPO | 0.077 | 0.046 | 0.068 | 0.001 | 0.722 |
| SCGB3A1_P103_R | SCGB3A1 | 0.004 | 0.020 | 0.004 | 0.002 | 0.722 |
| MEST_P4_F | MEST | 0.042 | 0.029 | 0.061 | 0.002 | 0.722 |
| PWCR1_P357_F | PWCR1 | 0.917 | 0.920 | 0.890 | 0.003 | 0.722 |
| NTRK3_P636_R | NTRK3 | 0.009 | 0.009 | 0.004 | 0.003 | 0.722 |
| TIE1_E66_R | TIE1 | 0.203 | 0.161 | 0.153 | 0.006 | 1.000 |
| HLA_DPA1_P205_R | HLA | 0.065 | 0.041 | 0.052 | 0.007 | 1.000 |
| EDNRB_P148_R | EDNRB | 0.995 | 0.995 | 0.995 | 0.009 | 1.000 |
| COL1A2_P48_R | COL1A2 | 0.033 | 0.023 | 0.028 | 0.011 | 1.000 |

### Validation of Selected CpG Sites in Phase II

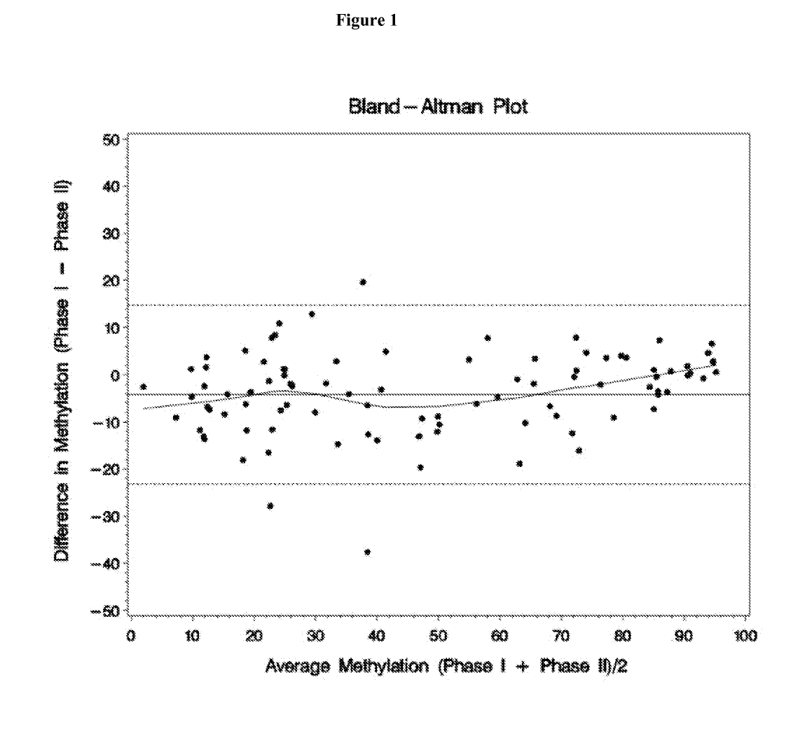

To validate the differentially methylated CpG sites identified in phase I within a larger number of patients and a broader range of demographic characteristics, a custom VeraCode methylation assay (Illumina, Inc.) was designed, and 96 of the 110 significant CpG sites were examined in 240 PaC cases and 240 matched controls. The 96 CpG sites were selected according to FDR values and median differences between cases and controls. Among the 480 subjects, 40 phase I subjects (20 cases and 20 controls) were included in order to compare the degree of agreement between the two methylation assays. Bland Altman plots (Bland and Altman, Lancet, 1(8476):307-10 (1986)) showed little mean shift and constant variation of differences over the range of values (FIG. 1), demonstrating reasonable agreement between the two assays. The two assays were significantly correlated as expected among all 96 CpG sites (mean Spearman correlation coefficient r=0.95).

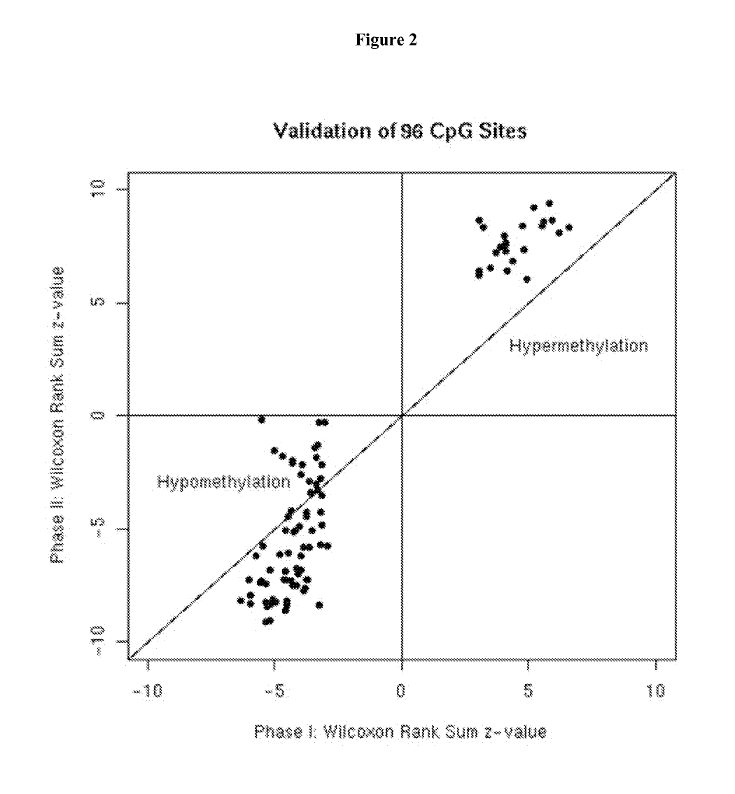

Among the 220 PaC patients who were unique to phase II, 47 patients were treated before blood was drawn. The methylation levels between these 47 treated cases and 173 never-treated cases were compared to evaluate the effect of treatment on the methylation status of these selected CpG sites. Two CpG sites (TAL1_P817 F and CSF3_E242_R) exhibited nominal differences (p=0.001 and 0.025, respectively), although these results could be due to chance, given the large number of comparisons. Overall, a significant treatment effect on the methylation of these selected CpG sites was not observed. Similarly, no effect was attributable to smoking history. Of the remaining 220 controls, five additional controls were excluded due to inadequate quality, leaving 215 controls who were unique to phase II (Table 2). A total of 173 never-treated cases and 215 controls were used for analysis in phase II. The Wilcoxon Rank Sum Test identified a significant difference (p<0.05) in 88 of the 96 selected CpGs. Importantly, all 88 of these validated CpG sites in phase II also exhibited the same direction of methylation change as phase I (FIG. 2). Of those, 23 and 65 CpG sites demonstrated hypermethylation and hypomethylation in PaC patients, respectively. Table 3 lists the 10 most significant CpG sites in the phase II study (Table 5 contained statistics of the 96 CpG sites in both phases I and II).

TABLE 5
Summary statistics (median (min, max) of the 96 significantly differentially
methylated CpG sites by phase and case/control status.

| CpG | Controls | Cases | p-value* |
|---|---|---|---|
| | Phase I | | |
| ITK_P114_F | 83.37 (66.78, 92.51) | 90.06 (48.41, 97.28) | <1E−10 |
| LCN2_P86_R | 56.08 (32.4, 78.23) | 43.98 (5.71, 92.14) | 2.00E−10 |
| ITK_E166_R | 88.59 (71.81, 96.04) | 94.14 (50.72, 99.63) | 5.00E−10 |
| PECAM1_E32_R | 23.19 (12.47, 45.11) | 15.66 (1.16, 44.89) | 1.60E−09 |
| LMO2_E148_F | 38.85 (13.84, 64.09) | 27.04 (3.69, 77.03) | 2.30E−09 |
| IL10_P348_F | 60.26 (31.16, 79.7) | 45.97 (1.14, 88.97) | 2.50E−09 |
| LCK_E28_F | 81.14 (65.35, 90.81) | 86.84 (50.58, 96.12) | 3.60E−09 |
| RUNX3_P247_F | 78.37 (41.19, 91.2) | 86.72 (32.49, 96.71) | 5.90E−09 |
| LMO2_P794_R | 31.43 (10.99, 54.48) | 20.27 (0.43, 70.88) | 1.02E−08 |
| MMP14_P13_F | 47.21 (23.97, 77.03) | 34.72 (0.95, 81.85) | 2.27E−08 |
| CTLA4_E176_R | 90.98 (76.72, 97.1) | 94.27 (73.46, 99.51) | 2.43E−08 |
| SPI1_P48_F | 39.1 (13.89, 63.67) | 28.97 (0.46, 75.52) | 3.00E−08 |
| SLC22A18_P216_R | 35.3 (15.09, 60.06) | 24.88 (2.89, 71.26) | 3.22E−08 |
| RUNX3_P393_R | 82.38 (49.34, 92.26) | 88.77 (39.04, 97.27) | 3.27E−08 |
| TRIP6_P1090_F | 30.42 (8.07, 66.9) | 22.32 (3.25, 74.23) | 4.17E−08 |
| RARA_P1076_R | 22.76 (10.53, 47.06) | 16.02 (1.68, 48.24) | 8.70E−08 |
| PI3_P274_R | 75.78 (53.4, 91.48) | 65.96 (10.63, 95.55) | 8.85E−08 |
| ERCC3_P1210_R | 61.67 (38.27, 80.11) | 50.39 (18.34, 89.01) | 9.79E−08 |
| LCN2_P141_R | 72.86 (42.33, 87.3) | 64.16 (22.65, 94.55) | 1.16E−07 |
| RUNX3_E27_R | 89.46 (71.07, 98.1) | 93.35 (11.97, 99.64) | 1.87E−07 |
| TNFRSF1A_P678_F | 65.33 (47.4, 79.92) | 56.61 (23.05, 87.1) | 2.14E−07 |
| GFI1_P208_R | 19.6 (4.31, 46.99) | 12.75 (0, 36.77) | 2.28E−07 |
| CD9_P585_R | 33.34 (13.45, 50.34) | 26.51 (5.27, 56.39) | 2.32E−07 |
| MFAP4_P197_F | 22.82 (6.75, 55.77) | 16.41 (2.01, 56.84) | 2.96E−07 |
| AIM2_P624_F | 37.67 (17.86, 58.1) | 28.43 (2.03, 61.51) | 3.54E−07 |
| TRIP6_P1274_R | 43.57 (14.61, 74.3) | 32.95 (0.76, 76.92) | 5.36E−07 |
| CSF3R_P472_F | 33.44 (15.09, 55.24) | 23.61 (0.68, 69.4) | 6.91E−07 |
| ZAP70_P220_R | 28.41 (0.19, 47.82) | 35.31 (0, 59.95) | 8.47E−07 |
| GRB7_E71_R | 29.92 (11.39, 58.66) | 22.23 (5.27, 84.04) | 1.55E−06 |
| IFNG_E293_F | 76.11 (44.81, 90.56) | 82.66 (40.37, 99.16) | 1.57E−06 |
| LTA_P214_R | 79.91 (61.63, 93.55) | 85.5 (44.8, 94.89) | 2.13E−06 |
| SEPT9_P374_F | 24.56 (11.19, 48.39) | 17.19 (2.52, 59.09) | 2.55E−06 |
| CD9_P504_F | 13.81 (1.88, 28.33) | 8.02 (0.54, 39.44) | 3.19E−06 |
| SPI1_E205_F | 20.85 (1.87, 42.24) | 15.34 (1.34, 49.21) | 4.34E−06 |
| ZMYND10_P329_F | 4.65 (0, 21) | 2.35 (0, 18.04) | 4.53E−06 |
| CSF3R_P8_F | 20.73 (1.86, 41.89) | 14.64 (0.77, 42.8) | 4.54E−06 |
| CSF3_E242_R | 66.58 (49.37, 81.52) | 59.27 (21.44, 90.38) | 5.10E−06 |
| PECAM1_P135_F | 18.55 (0.47, 45.36) | 11.37 (0.19, 33.84) | 5.10E−06 |
| EMR3_E61_F | 15.65 (5.25, 34.99) | 11.63 (0.24, 41.7) | 6.43E−06 |
| STAT5A_P704_R | 16.81 (5.15, 46.21) | 12.17 (0.28, 41.17) | 6.52E−06 |
| MMP9_P189_F | 31.53 (1.9, 56.05) | 22.65 (0.4, 49.69) | 7.01E−06 |
| SLC5A5_E60_F | 45.17 (21.04, 74.82) | 37.67 (8.07, 76.8) | 8.56E−06 |
| CRIP1_P874_R | 13.49 (4.42, 29.09) | 10.29 (1.06, 25.26) | 1.33E−05 |
| SYK_P584_F | 38.57 (0.24, 59.13) | 30.97 (0.67, 68.67) | 1.34E−05 |
| APBA2_P227_F | 94.88 (84.03, 99.63) | 97.09 (84.71, 99.65) | 1.38E−05 |
| TM7SF3_P1068_R | 56.68 (23.59, 82.45) | 46.79 (16.31, 87.94) | 1.59E−05 |
| RAB32_E314_R | 1.98 (0.27, 11.26) | 0.91 (0, 9.5) | 1.72E−05 |
| TAL1_P817_F | 21.45 (0, 49.38) | 14.26 (0, 40.73) | 1.74E−05 |
| IGFBP5_P9_R | 12.66 (0.07, 29.2) | 8.85 (0, 27.87) | 1.90E−05 |
| HPN_P374_R | 9.66 (0.26, 23.76) | 7.17 (0, 26.79) | 2.53E−05 |
| RHOH_P953_R | 97.62 (72.16, 99.47) | 99.03 (76.43, 99.6) | 3.49E−05 |
| MPL_P62_F | 29.8 (2.41, 58.43) | 22.88 (0, 55.22) | 3.54E−05 |
| PADI4_E24_F | 17.86 (0.32, 38.35) | 11.31 (0, 41.96) | 3.83E−05 |
| AIM2_E208_F | 96.3 (80.67, 99.46) | 97.97 (80.23, 99.54) | 4.20E−05 |
| KRT1_P798_R | 83.01 (66.54, 93.3) | 85.93 (68.78, 93.73) | 4.20E−05 |
| GPR116_P850_F | 96.05 (89.31, 98.99) | 97.09 (90.05, 99.25) | 4.26E−05 |
| TIE1_E66_R | 21.18 (0.91, 42.26) | 15.33 (0.55, 51.7) | 4.79E−05 |
| HGF_E102_R | 19.94 (7.37, 42.41) | 15.13 (0.53, 50.53) | 5.46E−05 |
| PADI4_P1011_R | 69.9 (51.57, 81.11) | 73.91 (51.55, 88.23) | 5.53E−05 |
| GSTM2_P453_R | 59.15 (40.51, 83.4) | 53.81 (25.36, 79.44) | 6.80E−05 |
| NOTCH4_E4_F | 15.25 (1.14, 41.15) | 9.84 (0.61, 35.21) | 6.89E−05 |
| MMP8_E89_R | 64.58 (42.74, 83.76) | 55.81 (0, 85.68) | 7.64E−05 |
| HIC_1_sEq_48_S103_R | 27.61 (0.09, 70.36) | 19.73 (0, 80.7) | 9.84E−05 |
| IFNG_P459_R | 85.46 (65.44, 97.27) | 88.28 (53.34, 96.99) | 1.06E−04 |
| EPHA2_P203_F | 43.85 (25.91, 67.36) | 36.39 (6.07, 77.13) | 1.07E−04 |
| CD82_P557_R | 19.56 (0, 51.55) | 13.07 (0, 43.55) | 1.08E−04 |
| VAMP8_P241_F | 31.69 (11.54, 50.58) | 24.36 (0.94, 54.16) | 1.25E−04 |
| CD86_P3_F | 12.46 (0.32, 40.34) | 9.46 (0.29, 32.83) | 1.72E−04 |
| DHCR24_P652_R | 44.87 (14.92, 62.58) | 39 (14.67, 66.69) | 1.76E−04 |
| SPARC_P195_F | 14.27 (2.87, 32.85) | 11.31 (0.52, 39.17) | 1.83E−04 |
| IL1RN_P93_R | 94.2 (87.08, 99.47) | 95.59 (85.25, 99.49) | 2.17E−04 |
| IFNGR2_P377_R | 21.62 (7.95, 48.7) | 16.2 (0, 68.34) | 2.17E−04 |
| CARD15_P302_R | 9.02 (0.6, 28.97) | 5.96 (0, 27.3) | 2.69E−04 |
| BCL2L2_P280_F | 7.89 (0, 21.29) | 4.95 (0, 25.19) | 2.78E−04 |
| SLC22A18_P472_R | 83.47 (72.01, 94.27) | 80.33 (34.95, 93.43) | 3.56E−04 |
| CSF1R_E26_F | 66.55 (30.32, 88.93) | 58.76 (16.56, 86.36) | 4.34E−04 |
| CLDN4_P1120_R | 89.62 (78.16, 96.19) | 91.19 (76.74, 97.54) | 4.54E−04 |
| GRB7_P160_R | 60.07 (33.43, 80.13) | 51.85 (14.14, 95.94) | 6.39E−04 |
| AXL_E61_F | 7.82 (0, 23.86) | 5.17 (0, 42) | 7.93E−04 |
| ALOX12_E85_R | 47.85 (7.84, 90.76) | 37.95 (2.82, 84.99) | 7.98E−04 |
| TFPI2_P152_R | 8.68 (1.16, 25.69) | 7.23 (0, 19.55) | 8.43E−04 |
| AGXT_P180_F | 84.04 (54.05, 94.35) | 78.61 (30.32, 95.02) | 8.90E−04 |
| IL10_P85_F | 16.33 (2.21, 31.74) | 11.98 (0.6, 28.58) | 1.04E−03 |
| KCNK4_E3_F | 30.96 (16.67, 58.04) | 26.78 (12.25, 65.38) | 1.16E−03 |
| JAK3_P1075_R | 69.62 (43.74, 86.14) | 64.45 (39.85, 85.81) | 1.31E−03 |
| IL6_P213_R | 4.39 (0.3, 16.26) | 3.24 (0, 12.35) | 1.36E−03 |
| NOTCH4_P938_F | 77.66 (57.82, 90.79) | 80.6 (58.86, 92.06) | 1.36E−03 |
| PTPN6_P282_R | 17.03 (0, 39.81) | 12.82 (0, 52.14) | 1.39E−03 |
| MATK_P190_R | 10.43 (0.92, 26.11) | 6.55 (0, 40.44) | 1.52E−03 |
| CEACAM1_P44_R | 7.92 (2.35, 21.58) | 5.91 (0.12, 17.51) | 1.62E−03 |
| CASP10_P334_F | 12.55 (0.26, 42.94) | 9.29 (0, 39.33) | 1.66E−03 |
| FGF1_P357_R | 95.37 (87.76, 99.49) | 96.51 (86.05, 99.63) | 2.14E−03 |
| IL17RB_E164_R | 10.41 (0.69, 28.02) | 7.84 (0, 29.55) | 2.21E−03 |
| CPA4_E20_F | 83.5 (64.39, 93.13) | 86.45 (59.8, 99.29) | 2.28E−03 |
| PTHR1_P258_F | 62.11 (31.07, 81.92) | 66.79 (38.33, 85.98) | 2.50E−03 |
| ESR1_P151_R | 9.47 (0.17, 28.16) | 7.72 (0, 26.14) | 3.44E−03 |
| | Phase II | | |
| ITK_P114_F | 84.6 (4.07, 94.97) | 88.98 (65.48, 95.66) | <1E−10 |
| LCN2_P86_R | 59.1 (25.07, 89.12) | 49.93 (13.42, 87.19) | <1E−10 |
| ITK_E166_R | 88.85 (75.13, 97.07) | 92.99 (66.52, 97.68) | <1E−10 |
| PECAM1_E32_R | 28.51 (3.17, 58.59) | 22.11 (7.07, 47.24) | <1E−10 |
| LMO2_E148_F | 49.69 (12.63, 66.96) | 39.04 (9.57, 74.81) | <1E−10 |
| IL10_P348_F | 71.91 (30.76, 84.99) | 63.82 (18.98, 86.99) | <1E−10 |
| LCK_E28_F | 85.93 (66.63, 94.24) | 89.99 (69.32, 95.97) | <1E−10 |
| RUNX3_P247_F | 75.28 (46.66, 92.97) | 84.1 (44.68, 94.7) | <1E−10 |
| LMO2_P794_R | 37.54 (7.76, 66) | 30.27 (4.33, 67.54) | 6.00E−10 |
| MMP14_P13_F | 56.94 (1.91, 75.33) | 48.07 (14.03, 82.2) | <1E−10 |
| CTLA4_E176_R | 90.42 (70.84, 96.69) | 93.6 (75.8, 97.07) | <1E−10 |
| SPI1_P48_F | 0 (0, 65.26) | 0 (0, 66.39) | 8.73E−01 |
| SLC22A18_P216_R | 45.39 (3.56, 67.22) | 37.63 (11.09, 65.12) | <1E−10 |
| RUNX3_P393_R | 85.91 (60.74, 94.14) | 90.52 (59.59, 96.07) | <1E−10 |
| TRIP6_P1090_F | 49.69 (15.94, 78.31) | 42.87 (7.49, 71.8) | 7.20E−09 |
| RARA_P1076_R | 15.22 (2.19, 34.67) | 10.9 (2.49, 31.5) | <1E−10 |
| PI3_P274_R | 76.46 (11.61, 86.93) | 68.74 (33.03, 89.85) | <1E−10 |
| ERCC3_P1210_R | 63.04 (34.49, 76.41) | 53.35 (20.9, 81.21) | <1E−10 |
| LCN2_P141_R | 70.29 (6.04, 90.27) | 63.03 (26.51, 90.6) | <1E−10 |
| RUNX3_E27_R | 85.68 (59.92, 96.29) | 90.88 (64.9, 97.41) | <1E−10 |
| TNFRSF1A_P678_F | 69.85 (28.76, 83.56) | 62.92 (32.87, 84.15) | <1E−10 |
| GFI1_P208_R | 27.99 (2.92, 57.92) | 21.48 (2.67, 51.52) | <1E−10 |
| CD9_P585_R | 29.61 (4.66, 54.39) | 25.49 (12.88, 46.71) | <1E−10 |
| MFAP4_P197_F | 20.52 (9.22, 37.18) | 15.63 (5.54, 32.33) | <1E−10 |
| AIM2_P624_F | 24.47 (2.07, 47.94) | 18.36 (4.35, 43.95) | <1E−10 |
| TRIP6_P1274_R | 0 (0, 74.89) | 0 (0, 72.24) | 1.19E−01 |
| CSF3R_P472_F | 35.13 (12.28, 53.48) | 27.56 (6.94, 61.54) | <1E−10 |
| ZAP70_P220_R | 47.65 (2.73, 76.92) | 52.39 (33.54, 75.02) | 1.50E−09 |
| GRB7_E71_R | 36.02 (1.92, 61.12) | 29.79 (6.77, 62.93) | l.00E−09 |
| IFNG_E293_F | 70.66 (21.85, 87.7) | 77.72 (39.38, 91.66) | <1E−10 |
| LTA_P214_R | 81.58 (59.26, 94.06) | 86.32 (57.97, 94.15) | <1E−10 |
| SEPT9_P374_F | 0 (0, 56.25) | 0 (0, 58.96) | 7.74E−02 |
| CD9_P504_F | 31.4 (1.94, 58.89) | 23.61 (2.66, 54.55) | <1E−10 |
| SPI1_E205_F | 46.01 (1.94, 68.8) | 38.93 (2.04, 67.89) | <1E−10 |
| ZMYND10_P329_F | 8.93 (2.5, 36.09) | 7.42 (1.79, 27.24) | 3.35E−07 |
| CSF3R_P8_F | 27.34 (3.84, 54.47) | 21.74 (5.7, 52.03) | <1E−10 |
| CSF3_E242_R | 61.98 (40.79, 78.17) | 56.43 (33.01, 78.13) | <1E−10 |
| PECAM1_P135_F | 14.99 (3.05, 32.67) | 11.05 (3.26, 25.28) | <1E−10 |
| EMR3_E61_F | 20.71 (6.49, 38.41) | 15.09 (3.15, 38.97) | <1E−10 |
| STAT5A_P704_R | 30.55 (3.94, 52.04) | 22.97 (6.02, 52.51) | <1E−10 |
| MMP9_P189_F | 37.54 (3.11, 57.08) | 32.85 (7.59, 55.08) | 1.30E−09 |
| SLC5A5_E60_F | 51.28 (27.1, 74.6) | 47.82 (9.37, 68.78) | 7.29E−06 |
| CRIP1_P874_R | 20.02 (2.69, 42.34) | 17.73 (8.46, 34.07) | 2.48E−05 |
| SYK_P584_F | 44.33 (4.94, 64.17) | 37.47 (2.48, 68.35) | <1E−10 |
| APBA2_P227_F | 92.19 (84.38, 97.59) | 93.86 (74.03, 97.18) | <1E−10 |
| TM7SF3_P1068_R | 54.8 (24.74, 84.6) | 46.58 (9.06, 71.14) | <1E−10 |
| RAB32_E314_R | 3.97 (2.42, 15.73) | 3.82 (2.18, 10.52) | 5.02E−02 |
| TAL1_P817_F | 27.29 (5.59, 46.43) | 25.55 (9.32, 43.64) | 3.56E−02 |
| IGFBP5_P9_R | 21.5 (3.54, 43.93) | 18.17 (3.12, 48.74) | 3.03E−07 |
| HPN_P374_R | 20.25 (5.94, 66.95) | 16.97 (7.79, 77.97) | 3.46E−07 |
| RHOH_P953_R | 92.75 (63.97, 96.75) | 94.02 (77.66, 96.71) | 2.00E−10 |
| MPL_P62_F | 34.54 (4.53, 66.72) | 28.09 (9.17, 53.73) | <1E−10 |
| PADI4_E24_F | 24.96 (2.95, 47.22) | 20.03 (2.9, 47.69) | <1E−10 |
| AIM2_E208_F | 94.45 (77.86, 97.47) | 95.66 (83.76, 98.08) | <1E−10 |
| KRT1_P798_R | 89.5 (71.75, 93.92) | 91.48 (75.18, 94.92) | <1E−10 |
| GPR116_P850_F | 95.47 (90.14, 97.05) | 96.11 (92.96, 97.1) | <1E−10 |
| TIE1_E66_R | 29.29 (2.4, 51.91) | 23.94 (2.85, 47.27) | <1E−10 |
| HGF_E102_R | 23.18 (1.86, 41.47) | 19.87 (2.6, 39.95) | 1.12E−06 |
| PADI4_P1011_R | 76.61 (9.64, 87.91) | 81.19 (55.69, 89.2) | <1E−10 |
| GSTM2_P453_R | 64.77 (39.09, 86.35) | 62.7 (40.83, 76.29) | 9.83E−03 |
| NOTCH4_E4_F | 20.61 (6.52, 44.05) | 16.87 (3.08, 33.64) | 6.00E−10 |
| MMP8_E89_R | 70.66 (6.82, 86.44) | 65.54 (32.82, 78.69) | <1E−10 |
| HIC_l_sEq_48_S103_R | 19.78 (7.67, 47.09) | 18.19 (3.33, 55.88) | 3.11E−02 |
| IFNG_P459_R | 89.86 (64.27, 96.86) | 92.57 (66.81, 97.08) | <1E−10 |
| EPHA2_P203_F | 74.53 (3.66, 89.34) | 67.54 (30.76, 87.52) | <1E−10 |
| CD82_P557_R | 22 (2.73, 60.61) | 17.48 (2.62, 39) | 5.40E−09 |
| VAMP8_P241_F | 40.17 (2.11, 63.54) | 34.37 (14.11, 52.08) | <1E−10 |
| CD86_P3_F | 15.87 (2.39, 30.32) | 13.49 (2.9, 28.27) | 2.22E−05 |
| DHCR24_P652_R | 48.39 (23.78, 74.34) | 41.86 (17.02, 67.4) | <1E−10 |
| SPARC_P195_F | 14.5 (3.93, 36.44) | 13.39 (5.63, 37.38) | 7.57E−06 |
| IL1RN_P93_R | 94.3 (85.78, 97.34) | 95.28 (90.71, 97.45) | <1E−10 |
| IFNGR2_P377_R | 29 (2.85, 53.14) | 22.53 (3.82, 41.23) | <1E−10 |
| CARD15_P302_R | 19.22 (3.24, 45.09) | 14.16 (3.66, 36.68) | 5.70E−09 |
| BCL2L2_P280_F | 18.13 (2.77, 54.1) | 16.71 (2.46, 44.21) | 3.81E−03 |
| SLC22A18_P472_R | 86.1 (69.16, 93.29) | 84.96 (73.83, 91.2) | 6.48E−04 |
| CSF1R_E26_F | 74.92 (25.36, 90.91) | 70.85 (39.78, 91.21) | 3.45E−07 |
| CLDN4_P1120_R | 91.31 (73.07, 95.82) | 92.64 (77.49, 95.59) | <1E−10 |
| GRB7_P160_R | 72.13 (41.09, 88.74) | 71.11 (13.96, 92.63) | 1.56E−01 |
| AXL_E61_F | 15.47 (2.5, 40.29) | 13.19 (3, 40.16) | 2.54E−03 |
| ALOX12_E85_R | 54.6 (6.87, 85.62) | 51.44 (7.44, 88.52) | 6.15E−02 |
| TFPI2_P152_R | 13.78 (2.43, 29.91) | 13.21 (2.21, 29.07) | 1.97E−01 |
| AGXT_P180_F | 77.55 (44.53, 90.06) | 74.85 (33.53, 87.81) | 1.03E−03 |
| IL10_P85_F | 21.04 (2.06, 38.09) | 16.05 (5.04, 46.92) | <1E−10 |
| KCNK4_E3_F | 0 (0, 89.11) | 0 (0, 63.18) | 7.74E−01 |
| JAK3_P1075_R | 70.21 (46.11, 85.05) | 68.02 (43.53, 87.34) | 5.43E−03 |
| IL6_P213_R | 10.48 (2.53, 33.05) | 7.87 (2.03, 25.36) | 1.33E−08 |
| NOTCH4_P938_F | 77.74 (44, 87.97) | 82.18 (64.28, 89.48) | <1E−10 |
| PTPN6_P282_R | 23.29 (3.67, 71.66) | 18.9 (2.67, 72.99) | 1.96E−05 |
| MATK_P190_R | 10.37 (3.76, 38.93) | 8.33 (2.91, 26.71) | 1.35E−06 |
| CEACAM1_P44_R | 10.06 (3.35, 25.72) | 8.58 (2.81, 24.96) | 4.35E−04 |
| CASP10_P334_F | 22.91 (10.21, 49.38) | 21.77 (9.58, 47.71) | 2.88E−02 |
| FGF1_P357_R | 93.66 (77.45, 96.31) | 94.92 (84.85, 97.12) | <1E−10 |
| IL17RB_E164_R | 11.97 (2.77, 43.06) | 12.35 (2.96, 37.44) | 7.60E−01 |
| CPA4_E20_F | 88.05 (56.56, 93.9) | 90.57 (68.86, 97.31) | 1.00E−10 |
| PTHR1_P258_F | 64.33 (3.69, 83.88) | 68.73 (35.03, 83.62) | 4.00E−10 |
| ESR1_P151_R | 10.92 (2.9, 25.71) | 8.85 (2.34, 24.42) | 7.80E−09 |

### Building and Validation of the Prediction Model

To build prediction models based on phase I data, 43 of the 96 CpG sites that showed less than 5% median β differences between cases and controls or p-value≧0.001 (FDR>0.007) in phase I were excluded. These filter criteria were set for the following technical considerations. First, CpG sites with smaller methylation differences are prone to laboratory error due to technical limitations. Second, CpG sites with less significant p-values are less likely to be replicated in future studies. Based on 53 remaining CpG sites, models were built using L1 and L2 penalties as described above using the phase I data.

An effective model was chosen based on criteria of ROC AUC and parsimony. This model was then tested using the phase II data without the 40 subjects assayed in both phases for the agreement study. When considering all cases and all controls, a panel of five CpG sites (Model I: IL10_P348, LCN2_P86, ZAP70_P220, AIM2_P624, and TAL1_P817) was identified. These five CpG sites were the first five CpGs to enter and remain in the Lasso model and also had the five largest coefficients from the Ridge model. This five CpG-only model exhibited good discrimination between patients and controls (c-statistic=0.85 in phase I and 0.76 in phase II) based on the logistic regression model. When including covariates in the logistic regression model (age, sex, 1^{st} degree of family history of PaC, and ABO blood type), the discrimination was improved in phase I (c-statistic=0.89), but decreased in phase II (c-statistic=0.72). When re-estimating coefficients in phase II (re-fitting), the discrimination was improved, but not dramatically (c-statistic=0.77 for five CpGs only, 0.77 after inclusion of covariates) (Table 6). When including resectable patients only and all controls, one CpG site (Model II: LCN2_P86) was identified that appeared to discriminate for resectable disease (c-statistic=0.78 in phase I and 0.74 in phase II).

TABLE 6
Methylation-based predication models and Area Under the ROC Curve (AUC).

| | | Phase I | | | Phase II | | | Phase II - Re-fit | | |
|---|---|---|---|---|---|---|---|---|---|---|
| | | | | CpG + | | | CpG + | | | CpG + |
| Mod- | CpG | CpGs | CpG + | Covariates* + | CpGs | CpG + | Covariates* + | CpGs | CpG + | Covariates* + |
| els | Illumina ID | only | Covariates* | ABO** | only | Covariates* | ABO** | only | Covariates* | ABO** |
| All Cases and All | | 60 controls, 119 cases | | | 215 controls, 173 cases | | | 215 controls, 173 cases | | |
| Controls | | | | | | | | | | |
| I | IL10_P348 | 0.85 | 0.86 | 0.89 | 0.76 | 0.75 | 0.72 | 0.77 | 0.77 | 0.77 |
| | LCN2_P86 | | | | | | | | | |
| | ZAP70_P220 | | | | | | | | | |
| | AIM2_P624 | | | | | | | | | |
| | TAL1_P817 | | | | | | | | | |

| Resectable Cases and | | 60 controls, 31 cases | | | 215 controls, 58 cases | | | 215 controls, 58 cases | | |
|---|---|---|---|---|---|---|---|---|---|---|
| All Controls | | | | | | | | | | |
| II | LCN2_P86 | 0.78 | 0.79 | 0.82 | 0.74 | 0.67 | 0.64 | 0.73 | 0.73 | 0.73 |

*Covariates includes age, sex, 1st degree Family history of PaC.
**ABO-blood type of O and non-O.

The results provided herein demonstrate that epigenetic variation in leukocyte DNA, manifested by reproducible methylation differences, can be used as an early diagnostic marker for differentiating between pancreatic cancer patients and humans without pancreatic cancer (e.g., healthy humans). For example, a panel that includes the IL10_P348, LCN2_P86, ZAP70_P220, AIM2_P624, and TAL1_P817 CpG methylation sites can be used to identify pancreatic cancer patients. The results provided herein also demonstrate that the LCN2_P86 CpG methylation site is capable of identifying human patients with resectable pancreatic cancer.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for identifying a human as having pancreatic cancer, wherein said method comprises:
(a) determining whether or not nucleic acid obtained from a blood sample of a human comprises at least three methylation CpG sites that have an altered methylation status indicative of pancreatic cancer, wherein said at least three methylation CpG sites are selected from the group consisting of IL10_P348, LCN2_P86, ZAP70_P220, AIM2_P624, and TAL1_P817 CpG methylation sites, and
(b) classifying said human as having pancreatic cancer if said nucleic acid comprises said at least three methylation CpG sites that have an altered methylation status indicative of pancreatic cancer, and classifying said human as not having pancreatic cancer if said nucleic acid does not comprise said at least three methylation CpG sites that have an altered methylation status indicative of pancreatic cancer.

2. The method of claim 1, wherein said blood sample is a blood sample obtained from a human not subjected to a prior pancreas tissue biopsy.

3. The method of claim 1, wherein said method comprises determining whether or not nucleic acid obtained from said blood sample comprises at least four methylation CpG sites that have an altered methylation status indicative of pancreatic cancer.

4. The method of claim 3, wherein said at least four methylation CpG sites are selected from the group consisting of IL10_P348, LCN2_P86, ZAP70_P220, AIM2_P624, and TAL1_P817 CpG methylation sites.

5. The method of claim 1, wherein said method comprises determining whether or not nucleic acid obtained from said blood sample comprises at least five methylation CpG sites that have an altered methylation status indicative of pancreatic cancer.

6. The method of claim 5, wherein said at least five methylation CpG sites are selected from the group consisting of IL10_P348, LCN2_P86, ZAP70_P220, AIM2_P624, and TAL1_P817 CpG methylation sites.

7. A method for identifying a human as having pancreatic cancer, wherein said method comprises:
(a) detecting the presence of at least three methylation CpG sites that have an altered methylation status indicative of pancreatic cancer in nucleic acid obtained from a blood sample of a human, wherein said at least three methylation CpG sites are selected from the group consisting of IL10_P348, LCN2_P86, ZAP70_P220, AIM2_P624, and TAL1_P817 CpG methylation sites, and
(b) classifying said human as having pancreatic cancer based at least in part on the presence of said at least three methylation CpG sites that have an altered methylation status indicative of pancreatic cancer.

8. The method of claim 7, wherein said blood sample is a blood sample obtained from a human not subjected to a prior pancreas tissue biopsy.

9. The method of claim 7, wherein said method comprises detecting the presence of at least four methylation CpG sites that have an altered methylation status indicative of pancreatic cancer in said nucleic acid.

10. The method of claim 9, wherein said at least four methylation CpG sites are selected from the group consisting of IL10_P348, LCN2_P86, ZAP70_P220, AIM2_P624, and TAL1_P817 CpG methylation sites.

11. The method of claim 7, wherein said method comprises detecting the presence of at least five methylation CpG sites that have an altered methylation status indicative of pancreatic cancer in said nucleic acid.

12. The method of claim 11, wherein said at least five methylation CpG sites are selected from the group consisting of IL10_P348, LCN2_P86, ZAP70_P220, AIM2_P624, and TAL1_P817 CpG methylation sites.

13. A method for identifying a human as having resectable pancreatic cancer, wherein said method comprises:
(a) determining whether or not nucleic acid obtained from a blood sample of a human comprises hypomethylation of an LCN2_P86 methylation CpG site, and
(b) classifying said human as having resectable pancreatic cancer if said nucleic acid comprises said hypomethylation of said LCN2_P86 methylation CpG site, and classifying said human as not having resectable pancreatic cancer if said nucleic acid does not comprise said hypomethylation of said LCN2_P86 methylation CpG site.

14. A method for identifying a human as having resectable pancreatic cancer, wherein said method comprises:
(a) detecting hypomethylation of an LCN2_P86 methylation CpG site of nucleic acid obtained from a blood sample of a human, and
(b) classifying said human as having resectable pancreatic cancer based at least in part on said hypomethylation.
